SEQUENCE LISTING (35 sequences)

SEQ ID NO: 1 - DNA, 2698 bp, Homo Sapiens
catgccaaatggcactggggcttcgtgtcgtgccacagcgtggaccgaaaatgcggacacatgcaggctgcctctcctcgcaggcagaagccacacgcagacctagaccctttgcaccgcatccccttattcaatcgcgcacccgccacccttcgacagttcctctccctccaccccaaccccacgccgcgcgcgaggctggccctttaagagccccgccccgactccctcccccctcgcgtgactgcgagcccccgcgccgggccggggaatgggtcggctgggtggctgcgcgggcctccggtccttctcacgcaacgcctgggcaccgcgcctccgggccaggtggggcggggacgggccgcctgacctctgccccctagagggatgtcgccggcgcacgcaagctagccgggggtagggtgggggctccgcgccaggtgccccctccgtggtccctgggcccgagtctttccgtggccccccgccgccggatttctgtgctctgccaatcaaagcactagccaccccgggagccaagagggaccctcaagggccggtgggtcctggctggagggaccgcgcgttgcaatcagcactaaggcgatcctagaggctgcgaggagccgctagtgagcgctcagcgagcctgccccttcgccatccattccgatccttcaatcaagaggcgcgaacctcagctagtcgcccgggctctgggggacaggtccagccccgcggcgcctctggccttccggcccccgtgacctcagggctggggtcgcagcgcttctcacgcgagccgggactcagtaaccccgggaaggaggtcaccacggggcagccccgcccccgcctgccgagtcctggtaggctgtagcgctggggaggcatctgcacgcccagcgttccagtgggtgcaaaaatgacgaagaggagtccccgcgccccaggatggagcttcccgtaccctctcttcgggctgtcctgggacttctccctcaagccccctcctcggctgggttctgcactgcccttgggacgccttggaattgggacttccaggtgttcccagccctcacccctctatgtacaggcaccgagatgtgtcccatagtgggttcttgcccacccgaccccccacccccgccgccctccgccacctttctctccaatcccagagagaccagcccggttcaggctgcttctccctccatctcagctcgctccagggaaggaggcgtggccacacgtacaagcccgcctataaaggtgcagtacttcaccctcaccctgaaggtgacagttcttggatgttccctgatccttgtgatcccaggctccaagagtccacccttcccagctcagctcagtacctcaggtgagttgctgggggacttctggcttgccctttctctcccaataaaaggaacattttggtgcctccaggacttcttaggtagctacctgtctagcacctccaaaaagggaggctcagagtgtttttagtgaccaggcagtctagccccctagtggggaaactgaggccaggggaagaggaggacttgcccatggtcccacagctggtaccagacctctagatacagatggcatctcattcaggacttcaggacccaggctccagctccatcccctagtgagtgtctccctgcctaccctggggcttccccatcaaggccacctggcaggctggaatatgtgcagcccctccctcaggctttctgatgacaggggcttctccttgggtggacagggtggatggagggggtgggctgggttcttaccagctgtaccctgccctagcctaagaagctacccctggcagattttaccctcctaagggtggcttgtcagtgctgagatgtcctagacagctgggacaatagaggcagatctgtgcaggagtcccaggcctttcctatctcattgaccatcttctttgtcctttgctgggagacaatcagggtgacagattgccaactgcagggagctggaaataccagtccctaaaaactcaccagtcacatctcccttggcctcctaccatcttacaaaggctgcaggtccttgggatacccactgtgcagaaggggacaccatagcacaccaaagcctggcactgtcccctgttgactcagggatctagtgtgctttgatatttagcccctccaggaagcctccctccactataatacttgtggtaggaaccatccatctccctgtcttgtgaggttctcctgtggggagcctaactggtaagactgtcaggttccccacagcagatctgggttttctcttccctctggatccagctgggtactctgaactgagagatcttgtcttaccctctctcagatgttgaaattggaccccagaaaagtaaaatgtgcagtccaagatcactttgactagaatgttggtctactgacctctagtccagggtaacaggcagagatgcctgatatgttggagagagtggtttatgaatttaaacaccctttttaggtcaagcttacagagaaagtattgcctcagtttcctttcagtttagatccattcctgatttccctgattccagtctggggttttcttacagcctagtgggaaccttccatttattctctgctctctggtaacctgcaaaagggggaggtccaaactgttcattcattgagaa

SEQ ID NO: 2 - DNA, 1316 bp, Artificial Sequence
  chemically treated genomic DNA (Homo sapiens)
tatgttaaatggtattggggtttcgtgtcgtgttatagcgtggatcgaaaatgcggatatatgtaggttgttttttttcgtaggtagaagttatacgtagatttagattttttgtatcgtatttttttatttaatcgcgtattcgttatttttcgatagtttttttttttttattttaattttacgtcgcgcgcgaggttggttttttaagagtttcgtttcgatttttttttttttcgcgtgattgcgagttttcgcgtcgggtcggggaatgggtcggttgggtggttgcgcgggttttcggttttttttacgtaacgtttgggtatcgcgttttcgggttaggtggggcggggacgggtcgtttgatttttgttttttagagggatgtcgtcggcgtacgtaagttagtcgggggtagggtgggggtttcgcgttaggtgtttttttcgtggtttttgggttcgagttttttcgtggtttttcgtcgtcggatttttgtgttttgttaattaaagtattagttatttcgggagttaagagggatttttaagggtcggtgggttttggttggagggatcgcgcgttgtaattagtattaaggcgattttagaggttgcgaggagtcgttagtgagcgtttagcgagtttgttttttcgttatttatttcgattttttaattaagaggcgcgaattttagttagtcgttcgggttttgggggataggtttagtttcgcggcgtttttggtttttcggttttcgtgattttagggttggggtcgtagcgttttttacgcgagtcgggatttagtaatttcgggaaggaggttattacggggtagtttcgttttcgtttgtcgagttttggtaggttgtagcgttggggaggtatttgtacgtttagcgttttagtgggtgtaaaaatgacgaagaggagttttcgcgttttaggatggagtttttcgtatttttttttcgggttgttttgggattttttttttaagtttttttttcggttgggttttgtattgtttttgggacgttttggaattgggatttttaggtgtttttagtttttatttttttatgtataggtatcgagatgtgttttatagtgggtttttgtttattcgattttttattttcgtcgtttttcgttatttttttttttaattttagagagattagttcggtttaggttgtttttttttttattttagttcgttttagggaaggaggcgtggttatacgtataagttcgtttataaaggtgtagtattttatttttattttgaaggtgatagtttttggatg

SEQ ID NO: 3 - DNA, 531 bp, Artificial Sequence
  chemically treated genomic DNA (Homo sapiens)
ggtttgtttgggaggtaaggcggtaggcgttgtagaattgatgggaattgtggtataggtgggaaatttggtttaataaattttttattgatttaggggattattttttttgagttaagttttggtaagcggtcggcgaaatttataggttttttttttggttgcgtttttagtttttagtttttttcgttttagagatgttttaggagcggtttttcggtgtaggtaacgggtgttcgggcggtttcgttcgtcgtttagagtttggaagtcgttattgcggtttaggataattcggttacgcggtcggcgtcgatttcgtacgttggagttcgttgtcgtacggcgttggtagtcgggggtggtgtttgaagttaggcgttttttgttttttcgtcggttcgggtgttcggttcgcgtcgttaggttttgggattttaggtcgtttcgtttagtagttcgcgttttgttcggtgcgtttagcgttttcgttttttattttaaatttttattttttgtgtttttaggggg

SEQ ID NO: 4 - DNA, 19 bp, Artificial Sequence
  chemically treated genomic DNA (Homo sapiens)
ggtttgtttgggaggtaag

SEQ ID NO: 5 - DNA, 18 bp, Artificial Sequence
  chemically treated genomic DNA (Homo sapiens)
ccccctaaaaacacaaaa

SEQ ID NO: 6 - DNA, 18 bp, Artificial Sequence
  chemically treated genomic DNA (Homo sapiens)
gtttttttcgttttagag

SEQ ID NO: 7 - DNA, 18 bp, Artificial Sequence
  chemically treated genomic DNA (Homo sapiens)
gttttttttgttttagag

SEQ ID NO: 8 - DNA, 18 bp, Artificial Sequence
  chemically treated genomic DNA (Homo sapiens)
ttggtagtcgggggtggt

SEQ ID NO: 9 - DNA, 18 bp, Artificial Sequence
  chemically treated genomic DNA (Homo sapiens)
ttggtagttgggggtggt

SEQ ID NO: 10 - DNA, 500 bp, Artificial Sequence
  chemically treated genomic DNA (Homo sapiens)
gggtgaaagagtgagttttatttttaaaacgaataaataaaaaattttaaaaaataaaagaatttagttaagtgtaaaagttttttttgattttaggttttagtgagttatcggcggggttgggattcgaatttagtggaattagaatcgtgtaggttttataatttatttagattttagtaattttaggttagagggttatcgcgtttatgcgaggtcgggtgggcgggtcgttagtttcgttttggggaggggttcgcgttgttgattggttgtggtcggtaggtgaatttttagttaattagcggtacggggggcggtgtttcggggtttatttggttgtagttacgtatttttttttagtggcgtcggaattgtaaagtatttgtgagtttgcggaagttagtttagattttagttcgttttagttcggttcgattcgatcgtattcggcgtttgttttcgttcggcgttttcggttagttatgggtttttggagt

SEQ ID NO: 11 - DNA, 23 bp, Artificial Sequence
  chemically treated genomic DNA (Homo sapiens)
gggtgaaagagtgagttttattt

SEQ ID NO: 12 - DNA, 21 bp, Artificial Sequence
  chemically treated genomic DNA (Homo sapiens)
actccaaaaacccataactaa

SEQ ID NO: 13 - DNA, 18 bp, Artificial Sequence
  chemically treated genomic DNA (Homo sapiens)
attagaatcgtgtaggtt

SEQ ID NO: 14 - DNA, 18 bp, Artificial Sequence
  chemically treated genomic DNA (Homo sapiens)
attagaattgtgtaggtt

SEQ ID NO: 15 - DNA, 18 bp, Artificial Sequence
  chemically treated genomic DNA (Homo sapiens)
gttagtttcgttttgggg

SEQ ID NO: 16 - DNA, 18 bp, Artificial Sequence
  chemically treated genomic DNA (Homo sapiens)
gttagttttgttttgggg

SEQ ID NO: 17 - DNA, 966 bp, Artificial Sequence
  chemically treated genomic DNA (Homo sapiens)
ggtgtttgataagatttgaaggattttcggattttagagtattatttcggaacgtttggtatttttgtcgcgcgggtacggcgatttttttagttgttaggttagtttttgattttcgcgaggggtttcgtagtgttgtagggggaggttggggattcgaataaaggagtagttttttcgtcggtgttattattcgacgttggttttaaggttcggttagtttgtttaaagttggtataagtttgttttgtaaaataaaagaagggaaagggggaaggggattttggtatagatttggttcgatttggatataggttgggttgtaagttcgcggggatcgggtttagaggggtagtgttgggaacgtttttttcggaaattaattttttagggtatcgttttttttttatgcgtcgttttattttcgtcggagattaggtttcgcgggggttatcgtgtttatcgtttcgcggtcgttggttttgggttttcgttgttggtttttttttttttttttcgtatttttttttttttttgtttttttcgatttttttttcgtcgtttggtttttttttttttcgttttgtttttcgcgtttcggttcgcgcgagttagacgttcgggtagttttcggcgtagcgcggtcgtagtagtttttttttttcgtacggtgtgagcgttcgtcgcgtcgaggcggtcggagtttcgagttagtttcgcggtcgtcgtcgtttagatcggacgataggttatttcgtcgcgttcgttcgagttttcgtttcgtcgttaacgttataattatcgcgtacggttttttgatttcgtttagtattgatcgggagagtcggagcgagtttttcggggagtagcgatgcgatttttcgggacggtcggggtagcgtttttggcgttgttggttgcgttttgttcggcgagtcgggttttggaggaaaagaaaggtaaggg

SEQ ID NO: 18 - DNA, 21 bp, Artificial Sequence
  chemically treated genomic DNA (Homo sapiens)
ggtgtttgataagatttgaag

SEQ ID NO: 19 - DNA, 19 bp, Artificial Sequence
  chemically treated genomic DNA (Homo sapiens)
cccttacctttcttttcct

SEQ ID NO: 20 - DNA, 18 bp, Artificial Sequence
  chemically treated genomic DNA (Homo sapiens)
agtattgatcgggagagt

SEQ ID NO: 21 - DNA, 18 bp, Artificial Sequence
  chemically treated genomic DNA (Homo sapiens)
agtattgattgggagagt

SEQ ID NO: 22 - DNA, 18 bp, Artificial Sequence
  chemically treated genomic DNA (Homo sapiens)
gagtttttcggggagtag

SEQ ID NO: 23 - DNA, 18 bp, Artificial Sequence
  chemically treated genomic DNA (Homo sapiens)
gagttttttggggagtag

SEQ ID NO: 24 - DNA, 272 bp, Artificial Sequence
  chemically treated genomic DNA (Homo sapiens)
agaagttgtttattgattggtggatttcgtttggcgttaattaggaaaggggggcggggtagtagttggttttattgagtcgttattatcgcgaaaggtcggtttggttgcgatagtttgggtaagaggtgtaggtcggtttggttttttgttattcggagttgggtaagcgggtgggagaatagcgaagatagcgtgagtttgggtcgttgtttcgagtttttcgttcggttttttttgtcgattcgttacgtttgtttggatttaatttt

SEQ ID NO: 25 - DNA, 20 bp, Artificial Sequence
  chemically treated genomic DNA (Homo sapiens)
agaagttgtttattgattgg

SEQ ID NO: 26 - DNA, 20 bp, Artificial Sequence
  chemically treated genomic DNA (Homo sapiens)
aaaattaaatccaaacaaac

SEQ ID NO: 27 - DNA, 18 bp, Artificial Sequence
  chemically treated genomic DNA (Homo sapiens)
ttgttattcggagttggg

SEQ ID NO: 28 - DNA, 18 bp, Artificial Sequence
  chemically treated genomic DNA (Homo sapiens)
ttgttatttggagttggg

SEQ ID NO: 29 - DNA, 186 bp, Artificial Sequence
  chemically treated genomic DNA (Homo sapiens)
gtttaaggttgtttgtgttataaatacgcggtttatatgtcgcggtgatacggtgttttttgggttcggcgggatagataatatgaatgtgttttttaaacgttttaagttgtagggatagttttcggtttagtttcgttttcggaagcgttttcgttttcgatgttttttgtagttgggaggagg

SEQ ID NO: 30 - DNA, 25 bp, Artificial Sequence
  chemically treated genomic DNA (Homo sapiens)
gtttaaggttgtttgtgttataaat

SEQ ID NO: 31 - DNA, 19 bp, Artificial Sequence
  chemically treated genomic DNA (Homo sapiens)
cctcctcccaactacaaaa

SEQ ID NO: 32 - DNA, 16 bp, Artificial Sequence
  chemically treated genomic DNA (Homo sapiens)
ggtgatacggtgtttt

SEQ ID NO: 33 - DNA, 16 bp, Artificial Sequence
  chemically treated genomic DNA (Homo sapiens)
ggtgatatggtgtttt

SEQ ID NO: 34 - DNA, 18 bp, Artificial Sequence
  chemically treated genomic DNA (Homo sapiens)
tttgggttcggcgggata

SEQ ID NO: 35 - DNA, 18 bp, Artificial Sequence
  chemically treated genomic DNA (Homo sapiens)
tttgggtttggtgggata

(19) United States
(12) Patent Application Publication
(10) Pub. No.: US 2004/0048279 A1
(43) Pub. Date: Mar. 11, 2004

(54) Method for detecting methylation states for a toxicological diagnostic

(76) Inventors: Alexander Olek, Berlin (DE); Christian Piepenbrock, Berlin (DE); Kurt Berlin, Stahnsdorf (DE)

Correspondence Address: KRIEGSMAN & KRIEGSMAN, 665 FRANKLIN STREET, FRAMINGHAM, MA, 01702, US

(21) Appl. No.: 10/416,905

(22) PCT Filed: Nov. 8, 2001
(86) PCT No.: PCT/EP01/12951

(30) Foreign Application Priority Data
Nov. 14, 2000 (DE) 100 56 802.5

(51) Int. Cl. C12Q 1/68

(52) U.S. Cl. 435/6

(57) ABSTRACT

The present invention concerns a method for toxicological diagnosis. A DNA sample is taken from an organism or a cell culture, which has previously been subjected to a specific substance that is to be investigated for its toxicological effect. The DNA contained in this sample is chemically pretreated and the base sequence of a part of the modified DNA is determined. A methylation state characteristic for the sample or a characteristic methylation pattern is concluded from this. The effect of a substance on the organism or the cell culture is concluded by comparison with data of the methylation states of other samples and/or compared with other substances from a toxicological point of view.

### FIELD OF THE INVENTION

The levels of observation that have been well studied in molecular biology according to developments in methods in recent years include the genes themselves, the transcription of these genes into RNA and the translation to proteins therefrom. During the course of development of an individual, which gene is turned on and how the activation and inhibition of certain genes in certain cells and tissues are controlled can be correlated with the extent and nature of the methylation of the genes or of the genome. In this regard, pathogenic states are also expressed by a modified methylation pattern of individual genes or of the genome.

In the present invention, the methylation states of toxicologically relevant genes and the data determined thereby are combined to form methylation patterns. By comparison of the patterns obtained with appropriate reference samples, comprehensive prognostic information on the toxicological properties of substances can be made. In addition, a method will be presented, which makes possible to a great extent the analysis of methylation positions of the genes to be investigated.

### PRIOR ART

The toxicological evaluation of chemical substances is currently being conducted in particular by experiments on animals. Animal experiments are ethically problematical, time-consuming and expensive. In order to be better able to estimate the toxicological consequences of substances, methods of analyzing gene expression are increasingly utilized. Such approaches were previously essentially based on the analysis of mRNA. In particular, thousands of genes can be investigated in parallel with DNA chips for changes in their transcriptional activity. Specific toxicological parameters can be concluded from the modified gene expression (Stoughton R. et al., U.S. Pat. No. 6,132,969).

5-Methylcytosine is the most frequent covalently modified base in the DNA of eukaryotic cells. For example, it plays a role in the regulation of transcription, in genetic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

A relatively new method that in the meantime has become the most widely used method for investigating DNA for 5-methylcytosine is based on the specific reaction of bisulfite with cytosine, which, after subsequent alkaline hydrolysis, is then converted to uracil, which corresponds in its base-pairing behavior to thymidine. In contrast, 5-methylcytosine is not modified under these conditions. Thus, the original DNA is converted so that methylcytosine, which originally cannot be distinguished from cytosine by its hybridization behavior, can now be detected by “standard” molecular biology techniques as the only remaining cytosine, for example, by amplification and hybridization or sequencing. All of these techniques are based on base pairing, which is now fully utilized. The prior art, which concerns sensitivity, is defined by a method that incorporates the DNA to be investigated in an agarose matrix, so that the diffusion and renaturation of the DNA is prevented (bisulfite reacts only on single-stranded DNA) and all precipitation and purification steps are replaced by rapid dialysis. (Olek, A. et al., Nucl. Acids Res. 1996, 24, 5064-5066). Individual cells can be investigated by this method, which illustrates the potential of the method. Of course, up until now, only individual regions of up to approximately 3000 base pairs long have been investigated; a global investigation of cells for thousands of possible methylation analyses is not possible. Of course, this method also cannot reliably analyze very small fragments of small quantities of sample. These are lost despite the protection from diffusion through the matrix.

An overview of other known possibilities for detecting 5-methylcytosines can be derived from the following review article: Rein, T., DePamphilis, M. L., Zorbas, H., Nucleic Acids Res. 1998, 26, 2255.

The bisulfite technique has been previously applied only in research, with a few exceptions (e.g., Zechnigk, M. et al., Eur. J. Hum. Gen. 1997, 5, 94-98). However, short, specific segments of a known gene have always been amplified after a bisulfite treatment and either completely sequenced (Olek, A. and Walter, J., Nat. Genet. 1997, 17, 275-276) or individual cytosine positions are detected by a “primer extension reaction” (Gonzalgo, M. L. and Jones, P. A., Nucl. Acid Res. 1997, 25, 2529-2531, WO-Patent 95-00669) or an enzyme step (Xiong, Z. and Laird, P. W., Nucl. Acids Res. 1997, 25, 2532-2534). Detection by hybridization has also been described (Olek et al., WO-A 99-28,498).

Other publications which are concerned with the application of the bisulfite technique for the detection of methylation in the case of individual genes are: Xiong, Z. and Laird, P. W. (1997), Nucl. Acids Res. 25, 2532; Gonzalgo, M. L. and Jones, P. A. (1997), Nucl. Acids Res. 25, 2529; Grigg, S. and Clark, S. (1994), Bioassays 16, 431; Zeschnik, M. et al. (1997), Human Molecular Genetics 6, 387; Teil, R. et al. (1994), Nucl. Acids Res. 22, 695; Martin, V. et al. (1995), Gene 157, 261; WO-A 97-46,705, WO 95-15,373 and WO 45,560.

A review of the prior art in oligomer array production can be taken from a special edition of Nature Genetics that appeared in January 1999 (Nature Genetics Supplement, Volume 21, January 1999) and the literature cited therein.

Probes with multiple fluorescent labels have been used for scanning an immobilized DNA array. Particularly suitable for fluorescent labels is the simple introduction of Cy3 and Cy5 dyes at the 5-OH of the respective probe. The fluorescence of the hybridized probes is detected, for example, by means of a confocal microscope. The dyes Cy3 and Cy5, among many others, are commercially available.

Matrix-assisted laser desorptions/ionization mass spectrometry (MALDI-TOF) is a very powerful development for the analysis of biomolecules (Karas, M. and Hillenkamp, F. (1988), Laser desorption ionization of proteins with molecular masses exceeding 10000 daltons. Anal. Chem. 60: 2299-2301). An analyte is embedded in a light-absorbing matrix. The matrix is vaporized by a short laser pulse and the analyte molecule is transported unfragmented into the gaseous phase. The analyte is ionized by collisions with matrix molecules. An applied voltage accelerates the ions in a field-free flight tube. Ions are accelerated to varying degrees based on their different masses. Smaller ions reach the detector sooner than large ions.

Genomic DNA is obtained from DNA of cells, tissue or other test samples by standard methods. This standard methodology is found in references such as Fritsch and Maniatis, eds., Molecular Cloning: A Laboratory Manual, 1989.

At present, it is not state of the art to investigate large quantities of samples relative to more important methylation positions for toxicological diagnosis.

### PRESENTATION OF THE PROBLEM

The present invention will present a method, which is suitable for the diagnosis of methylation states of genes with toxicological relevance. The invention is based on the knowledge that cytosine methylation states are particularly suitable for the diagnosis of changes in the expression of genes with toxicological relevance.

### DESCRIPTION

The present invention describes a method for evaluating toxicological properties of specific substances. The method is based on the detection of specific changes in the methylation state or methylation pattern of genomic DNA, which [changes] occur due to the substance being tested.

A methylation state in this invention is a state of methylation of cytosine bases that is specific for a DNA sample.

A sample that contains the DNA of an organism or a cell culture, which [sample] has been derived from this organism or cell culture, was previously subjected to a substance to be tested for its toxicological effect.

The genomic DNA to be analyzed is thus preferably obtained from the usual sources for DNA, such as, e.g., cell lines, blood, sputum, stool, urine, cerebrospinal fluid, tissue embedded in paraffin, for example tissue from eyes, intestine, kidney, brain, heart, prostate, lungs, breast or liver, histological slides and all possible combinations thereof.

In a first step of the method, the DNA that is obtained is treated in such a way that methylated cytosine bases produce a different base sequence in said DNA. Then the base sequence of said treated DNA sample is determined and a conclusion is made of a methylation state or a methylation pattern that is characteristic for the sample. Finally, by comparison of the methylation state or methylation pattern obtained with the methylation states or methylation patterns of other samples, a conclusion can be made of the effect of the substance used on the organism or cell culture and/or the effects of different substances can be compared with one another.

Preferably, in the first step of the method, a genomic DNA sample is chemically treated in such a way that cytosine bases that are unmethylated at the 5′-position are converted to uracil, thymine or another base unlike cytosine in its hybridization behavior. In the following, this is understood as chemical pretreatment.

The above-described treatment of genomic DNA with bisulfite (hydrogen sulfite, disulfite) and subsequent alkaline hydrolysis, which leads to a conversion of unmethylated cytosine nucleobases to uracil is preferred for this purpose.

In the second step of the method, the base sequence of a part of the chemically treated DNA is determined and a conclusion is made of the methylation states characteristic of the sample.

Preferably, in a second step of the method, fragments of the chemically pretreated genomic DNA are first amplified with the use of primer oligonucleotides. Preferably, more than 10 different fragments, which are 100-2000 base pairs long, are amplified.

In a preferred variant of the method, the amplification is preferably conducted by means of the polymerase chain reaction (PCR), wherein a heat-stable DNA polymerase is preferably used.

It is preferred according to the invention that the amplification of several DNA segments is conducted in one reaction vessel.

In a preferred variant of the method, the set of primer oligonucleotides comprises at least two oligonucleotides, each of whose sequences is inversely complementary or identical to a segment that is at least 18 base pairs long of the sequences of the genes to be investigated. The primer oligonucleotides are preferably characterized in that they do not contain a CpG dinucleotide.

Preferably, at least one of the two primer oligonucleotides used for amplifying a specific segment of the chemically pretreated DNA contains identifiable labels.

It is preferred according to the invention that the labels of the amplificates are fluorescent labels.

It is preferred according to the invention that the labels of the amplificates are radionuclides.

It is preferred according to the invention that the labels of the amplificates are removable molecular fragments with typical masses, which are detected in a mass spectrometer.

According to the invention, it is preferred that the amplificates, fragments of the amplificates or probes that are complementary to the amplificates, are detected in the mass spectrometer.

It is preferred according to the invention that the produced fragments have a single positive or single negative net charge for better detectability in the mass spectrometer.

It is preferred according to the invention that the detection is carried out and visualized by means of matrix-assisted laser desorption/ionization mass spectrometry (MALDI) or by means of electrospray mass spectrometry (ESI).

It is preferred according to the invention that at least one primer oligonucleotide is bound to a solid phase in the amplification.

Preferably, the probe oligonucleotides or PNA oligomers are bound to defined sites of a solid phase.

According to the invention it is further preferred that different oligonucleotide and/or PNA oligomer sequences are arranged on a planar solid phase in the form of a rectangular or hexagonal grid.

The solid-phase surface is preferably comprised of silicon, glass, polystyrene, aluminum, steel, iron, copper, nickel, silver, or gold.

In the third step of the method, the amplificates are hybridized to a set of at least 10 oligonucleotide or PNA oligomer probes. The amplificates thus serve as samples, which hybridize to the oligonucleotides previously bound to a solid phase. In the sense of this invention, hybridization is to be understood as a binding with the formation of a duplex structure of an oligonucleotide to a completely complementary sequence in the sense of a Watson-Crick base pairing in the sample DNA. The unhybridized fragments are then removed.

Said oligonucleotides comprise at least one base sequence with a length of 13 nucleotides, which is inversely complementary or identical to a segment of the base sequences of the genes to be investigated. The cytosine of the CpG dinucleotide is the 5th to 9th nucleotide viewed from the 5′-end of the 13-mer. One oligonucleotide is present for each CpG dinucleotide.

Said PNA oligomers comprise at least one base sequence with a length of 9 nucleotides, which is inversely complementary or identical to a segment of the base sequences of the genes to be investigated, which contains at least one CpG dinucleotide. The cytosine of the CpG dinucleotide is the 4th to 6th nucleotide viewed from the 5′-end of the 9-mer. One oligonucleotide is present for each CpG dinucleotide.

In the fourth step of the method, the unhybridized amplificates are removed.

In the last step of the method, the hybridized amplificates are detected.

It is preferred according to the invention that labels which are introduced on the amplificates at any position of the solid phase at which an oligonucleotide sequence is found, can be identified. The use of a method for the diagnosis of methylation states within a group of genes, which are characterized by a particularly well-documented association with toxicological processes, is preferred according to the invention.

The method preferably serves for the diagnosis and/or prognosis of adverse events for patients or individuals, whereby these adverse events are related to the diagnosis of toxicologically important parameters.

According to the invention, the method is used for the diagnosis and/or prognosis of adverse events for patients or individuals, whereby these adverse events are related to the diagnosis of toxicologically important parameters.

The set of genes to be investigated comprises at least one of the genes listed in Table 1 or, however, sequences which are identical, or at least 85% homologous in the region of the exon, to the genes listed in Table 1.

| | GenBank |
|---|---|
| Gene Name | Accession #(s) |
| serotransferrin precursor; siderophilin; beta-1-metal binding globulin | M12530 |
| lactotransferrin precursor; lactoferrin | X53961 |
| apolipoprotein E precursor (APOE) | M12529 |
| lipopolysaccharide-binding protein precursor (LBP) | M35533 |
| B-lymphocyte kinase; tyrosine-protein kinase BLK; p55-BLK | Z33998 |
| apolipoprotein A-I precursor (APOAI) | X00566 |
| apolipoprotein A-II precursor (APOAII) | X00955 |
| apolipoprotein C-III precursor (APOCIII) | X01388 |
| endothelin 1 (ET1) | Y00749 |
| macrophage colony stimulating factor 1 (CSF1; MCSF) | M37435 |
| familial intrahepatic cholestasis 1 protein (FIC1) | AF038007 |
| vascular endothelial growth factor D (VEGFD); C-FOS-induced growth factor (FIGF) | D89630 |
| complement component 4-binding protein alpha (C4B-binding protein; C4BPA); | |
| proline-rich protein (PRP) | M31452 |
| insulin-like growth factor II (IGF2); somatomedin A | M29645 |
| granulocyte-macrophage colony stimulating factor (GM-CSF); CSF2 | M11220 |
| epidermal growth factor precursor (EGF); beta-urogastrone | X04571 |
| hepatocyte growth factor activator (HGF activator) | D14012 |
| macrophage inflammatory protein 1 beta precursor (MIP1-beta); | |
| T-cell activation protein 2 (AT2); PAT 744; H400; SIS-gamma; | |
| lymphocyte activation gene 1 protein (LAG 1); HC21; | |
| small inducible cytokine A4 (SCYA4); G 26 T-lymphocyte secreted protein | J04130 |
| glial growth factor 2 precursor (GGFHPP2); neuregulin; heregulin-beta3 + neu | |
| differentiation factor + heregulin-alpha | L12260; |
| | L12261 + |
| | U02326 + M94165 |
| T-cell-specific rantes protein precursor; sis delta; small inducible cytokine A5 (SCYA5); | |
| rantes pro-inflammatory cytokine | M21121 |
| macrophage inflammatory protein 1 alpha precursor (MIP1-alpha); | |
| tonsillar lymphocyte LD78 alpha protein; G0S19-1 protein; PAT 464.2; SIS-beta; | |
| small inducible cytokine A3 (SCYA3) | M23452 |
| oncostatin M (OSM) | M27288 |
| insulin-like growth factor binding protein 1 (IGFBP1); placental protein 12 (PP12) | M31145 |
| vascular endothelial growth factor precursor (VEGF); | |
| vascular permeability factor (VPF) | M32977; M27281 |
| hepatocyte growth factor (HGF); scatter factor (SF); hepatopoeitin A | M60718 |
| thymosin beta-10 (TMSB10; THYB10); PTMB10 | M92381 |
| interferon gamma-induced protein precursor (gamma-IP10) | X02530 |
| macrophage inflammatory protein 2 alpha (MIP2-alpha); | |
| growth-regulated protein beta (GRO-beta) | X53799 |
| OX40 ligand (OX40L); GP34; tax-transcriptionally activated glycoprotein 1 (TXGP1) | X79929 |
| transforming growth factor-beta 3 (TGF-beta3) | J03241 |
| delta-like protein precursor (DLK) | U15979; Z12172 |
| insulin-like growth factor IA precursor (IGF1A); IGFBP1; | |
| somatomedin C + insulin-like growth factor I (IGF1) | M27544 + M37484 |
| CC chemokine eotaxin precursor; eosinophil chemotactic protein; | |
| small inducible cytokine A11 (SCYA11) | D49372; |
| | Z75669; Z75668 |
| sonic hedgehog (SHH) | L38518 |
| interleukin-1 receptor antagonist protein precursor (IL-1RA; IRAP) | M63099 |
| macrophage inhibitory cytokine 1 (MIC1) | AF019770 |
| erythropoietin | M11319 |
| eosinophil granule major basic protein precursor (MBP); | |
| pregnancy-associated major basic protein; bone marrow proteoglycan 2 | Y00809 |
| insulin-like growth factor-binding protein 3 precursor | |
| (IGF-binding protein 3; IGFBP3; IBP3) | M31159; M35878 |
| cellular retinoic acid-binding protein II (CRABP2) | M68867 |
| corticoliberin precursor; corticotropin-releasing factor (CRF); | |
| corticotropin releasing hormone (CRH) | V00571 |
| interferon gamma precursor (IFN-gamma; IFNG); immune interferon | X01992; M29383 |
| interleukin-2 precursor (IL-2); T-cell growth factor (TCGF) | A14844 |
| interleukin-1 alpha precursor (IL-1 alpha; IL1A); hematopoietin-1 | X02851 |
| interleukin-4 precursor (IL-4); B-cell stimulatory factor 1 (BSF-1); | |
| lymphocyte stimulatory factor 1 | M13982 |
| interleukin-6 precursor (IL-6); B-cell stimulatory factor 2 (BSF2); | |
| interferon beta-2 (IFNB2); hybridoma growth factor | X04602; M14584 |
| interleukin-5 precursor (IL-5); T-cell replacing factor (TRF); | |
| eosinophil differentiation factor; B-cell differentiation factor I | X04688; J03478 |
| interleukin-12 beta subunit precursor (IL-12B); | |
| cytotoxic lymphocyte maturation factor 40-kDa subunit (CLMF p40); | |
| NK cell stimulatory factor subunit 2 (NKSF2) | M65290 |
| interleukin-12 alpha subunit precursor (IL-12A); | |
| cytotoxic lymphocyte maturation factor 35-kDa subunit (CLMF p35); | |
| NK cell stimulatory factor subunit 1 (NKSF1) | M65291 |
| pancreatitis-associated protein 1 precursor | D13510 |
| alpha-1-acid glycoprotein 1 precursor (AGP1); orosomucoid 1 (OMD1) | X02544 |
| C-reactive protein precursor | X56692 |
| corticosteroid-binding globulin | J02943 |
| prostaglandin-endoperoxide synthase 1 precursor; prostaglandin G/H synthase 1 | |
| (PGH synthase 1; PTGS1; PHS1); cyclooxygenase 1 (COX1) | M59979 |
| amphiphysin (AMPH) | U07616 |
| 5-hydroxytryptamine 1D receptor (5-HT-1D; HTR1D); serotonin receptor | M89955 |
| neuromedin B precursor | M21551 |
| major prion protein precursor (PRP); PRP27-30; PRP33-35C; ASCR | M13667 |
| dopamine beta-hydroxylase (DBH); dopamine-beta-monooxygenase precursor | X13255 |
| Alzheimer's disease amyloid A4 protein precursor; protease nexin-II (PN-II); APPI | Y00264 |
| membrane-bound & soluble catechol-O-methyltransferase (COMT) | M65212 |
| flavin-containing amine oxidase A; monoamine oxidase (MAO-A) | M68840 |
| erythropoietin receptor (EPOR) | M60459 |
| cation-independent mannose-6-phosphate receptor precursor | |
| (CI man-6-P receptor; CI-MPR); | |
| insulin-like growth factor II receptor (IGFR II) | Y00285; J03528 |
| activin receptor type II precursor (ACTRIIA; ACVR2) | D31770 |
| RETINOID X RECEPTOR GAMMA (RXR-GAMMA) | U38480 |
| transcriptional enhancer factor (TEF1); protein GT-IIC; transcription factor 13 (TCF13) | M63896 |
| glucocorticoid receptor (GRL) | M10901 |
| orphan nuclear hormone receptor BD73 | L31785 |
| low-density lipoprotein receptor (LDL receptor; LDLR) | M28219 |
| sulfonylurea receptor 2A (SUR2A) | AF061323 |
| sulfonylurea receptor (SUR); | |
| ATP-binding cassette subfamily C (CFTR/MRP) member 8 (ABCC8) | L78207 |
| farnesol receptor HRR-1 | U68233 |
| tyrosine protein kinase receptor UFO | X66029 |
| colorectal cancer suppressor protein precursor (DCC) | X76132 |
| vascular cell adhesion protein 1 | X53051 |
| alpha1 catenin (CTNNA1); cadherin-associated protein; | |
| alpha E-catenin | D13866; D14705; |
| | L23805; L22080 |
| integrin alpha 9 (ITGA9); integrin alpha-RLC | D25303; L24158 |
| intercellular adhesion molecule 1 precursor (ICAM1); major group rhinovirus receptor; | |
| CD54 antigen | J03132 |
| ras-related protein RAB5A | M28215 |
| E-selectin precursor (SELE); endothelial leukocyte adhesion molecule 1 (ELAM1); | |
| leukocyte-endothelial cell adhesion molecule 2 (LECAM2); CD62E antigen | M30640 |
| NADH-ubiquinone dehydrogenase 1 beta subcomplex 7 18-kDa subunit (NDUFB7); | |
| complex I-B18 (CI-B18); cell adhesion protein SQM1 | M33374 |
| neural-cadherin precursor (N-cadherin; NCAD); | |
| cadherin 2 (CDH2) | M34064; X57548; |
| | X54315; S42303 |
| cell surface adhesion glycoproteins LFA-1/CR3/p150,95 beta-subunit precursor; | |
| LYAM1; integrin beta 2 (ITGB2); CD18 antigen; complement receptor C3 beta subunit | M15395 |
| fibronectin receptor alpha subunit (FNRA); integrin alpha 5 (ITGA5); VLA5; | |
| CD49E antigen | X06256 |
| fibronectin receptor beta subunit (FNRB); integrin beta 1 (ITGB1); | |
| very late antigen 4 beta subunit (VLA4); CD29 antigen | X07979 |
| integrin alpha L (ITGAL); leukocyte adhesion glycoprotein alpha subunit precursor; | |
| leukocyte function-associated molecule 1 alpha chain (LFA1); CD11A antigen | Y00796 |
| cadherin 6 precursor (CDH6); kidney cadherin (K-cadherin) | D31784 |
| cadherin 11 precursor (CDH11); osteoblast-cadherin (OB-cadherin); OSF4 | L34056 |
| cadherin 12 (CDH12); brain cadherin precursor (Br-cadherin); | |
| neural cadherin 2 (N-cadherin 2) | L34057; L33477 |
| cadherin 13 (CDH13); truncated cadherin precursor (T-cadherin); | |
| heart cadherin (H-cadherin) | L34058; |
| | U59289; U59288 |
| cadherin 3 (CDH3); placental cadherin precursor (P-cadherin; CDHP) | X63629 |
| GAP JUNCTION ALPHA-5 PROTEIN (CONNEXIN 40) (CX40) | L34954 |
| INVOLUCRIN | M13903 |
| fibrinogen G gamma polypeptide | X51473; |
| | X02415 K02569 |
| plasma-cell membrane glycoprotein PC-1; alkaline phosphodiesterase I; | |
| nucleotide pyrophosphatase (NPPase) | M57736 |
| annexin V; lipocortin V; endonexin II; calphobindin I (CBP-I); | |
| placental anticoagulant protein I (PAP-I); PP4; thromboplastin inhibitor; | |
| vascular anticoagulant-alpha (VAC-alpha; anchorin CII | X12454 |
| laminin alpha 1 subunit precursor (LAMA1); laminin A chain | X58531 |
| intestinal fatty acid-binding protein 2 (FABP2; IFABP)+ | |
| liver fatty acid-binding protein 1 (FABP1; LFABP) | M10050 + M10617 |
| sodium-independent organic anion transporter; | |
| organic anion transporting polypeptide (OATP); SLC21A3 | U21943 |
| polyspecific organic cation transporter N1 (OCTN1) | AB007448 |
| TNF-alpha-stimulated ABC protein (TSAP) | AF027302 |
| organic cation transporter-like protein 2 (ORCTL2) | AF037064 |
| organic cation transporter N2 (OCTN2) | AF057164 |
| MRP/organic anion transporter (MOAT-B) | AF071202 |
| adrenoleukodystrophy-related protein (ALDR) | AJ000327 |
| skeletal muscle adenine nucleotide translocator 1 (ANT1); | |
| heart/skeletal muscle ADP/ATP carrier protein isoform T1; | |
| ADP/ATP translocase 1 | J02966 |
| down-regulated in adenoma protein (DRA) | L02785 |
| mitochondrial uncoupling protein 3 (UCP3) | AF011449 |
| mitochondrial carnitine palmitoyltransferase II precursor (CPTase; CPT2) | M58581 |
| mitochondrial brown fat uncoupling protein 1 (UCP1) | U28480 |
| prostaglandin transporter (PGT); solute carrier family 21 member 2 (SLC21A2) | U70867 |
| mitochondrial uncoupling protein 2 (UCP2); UCPH | U82819 |
| bile salt export pump (BSEP) | AF091582 |
| anthracycline resistance-associated protein (ARA) | X95715 |
| kidney organic cation transporter | X98333 |
| multidrug resistance-associated protein 3 (MRP3); MLP2; ABCC3 | Y17151 |
| antigen peptide transporter 2 (APT2); peptide supply factor 2 (PSF2); | |
| peptide transporter involved in antigen processing 2 (TAP2); | |
| ATP-binding cassette subfamily B (MBR/TAP) member 3 (ABCC3); | |
| HLA class II histocompatibility antigen DO beta chain precursor | X66401; |
| | L09191; L10287 |
| putative renal organic anion transporter 1 (hROAT1) | AF057039 |
| chloride conductance regulatory protein ICLN; nucleotide-sensitive chloride channel 1A; | |
| chloride ion current inducer protein (CLCI); reticulocyte PICLN | X91788 |
| neutral amino acid transporter A (SATT); | |
| alanine/serine/cysteine/threonine transporter (ASCT1) | L14595 |
| monocarboxylate transporter 1 (MCT1) | L31801 |
| ileal sodium-dependent bile acid transporter (ISBT); | |
| ileal sodium/taurocholate cotransporting polypeptide (NTCP2); SLC10A2 | U10417 |
| sodium-dependent bile acid cotransporter; | |
| hepatic sodium/taurocholate cotransporting polypeptide (NTCP); SLC10A1 | L21893 |
| sodium- & chloride-dependent glycine transporter 1 (GLYT-1) | S70609 |
| cystic fibrosis transmembrane conductance regulator (CFTR); | |
| cAMP- dependent chloride channel | M28668 |
| canalicular multispecific organic anion transporter; | |
| multidrug resistance-associated protein 2 (MRP2); | |
| canalicular multidrug resistance protein | U63970 |
| organic cation transporter 1 | U77086 |
| GAP JUNCTION BETA-1 PROTEIN (CONNEXIN 32) | |
| (CX32) (LIVER GAP JUNCTION PROTEIN) | X04325 |
| cadherin1 (CDH1); epithelial cadherin precursor (E-cadherin; CDHE); | |
| uvomorulin (UVO); CAM 120/80 | Z13009 |
| smoothened; GX | U84401 |
| ephrin type-A receptor 2 precursor; epithelial cell kinase (ECK); | |
| tyrosine-protein kinase receptor ECK | M59371 M36395 |
| NADPH-cytochrome p450 reductase | S90469 |
| NCK melanoma cytoplasmic src homolog (HSNCK) | X17576 |
| JV18-1. HMAD-2 OR MADR2 OR SMAD2 | U68018 |
| dual-specificity mitogen-activated protein kinase kinase 1 (MAP kinase kinase 1; | |
| MAPKK 1; MKK1); extracellular signal-regulated kinase 1; | |
| ERK activator kinase 1 | L05624 |
| c-jun N-terminal kinase 1 (JNK1); JNK46 | L26318 |
| mitogen-activated protein kinase p38 (MAP kinase p38); | |
| cytokine suppressive anti-inflammatory drug-binding protein | |
| (CSAID binding protein; CSBP); MAX-interacting protein 2 (MXI2) | L35253; L35263 |
| protein kinase C beta I (PKC-beta-1) | M27545; X06318 |
| mitogen-activated protein kinase 9 (MAP kinase 9; MAPK9; PRKM9); | |
| c-jun N-terminal kinase 2 (JNK2); JNK55 | L31951 |
| C-jun N-terminal kinase 3 alpha2 (JNK3A2); | U34819 + U07620 |
| PRKM10 + MAP kinase p493F12 | |
| dual-specificity mitogen-activated protein kinase kinase 6 | |
| (MAP kinase kinase 6; MAPKK 6; MKK6); MAPK/ERK kinase 6; SAPKK3 | U39657 |
| p21-activated kinase gamma (PAK-gamma; PAK2); PAK65; S6/H4 kinase | U24153 |
| mitogen-activated protein kinase P38 beta (MAP kinase P38 beta); | |
| stress-activated protein kinase 2 (SAPK2) | U53442 |
| MAPK/ERK kinase kinase 3 (MEK kinase 3; MEKK3) | U78876 |
| dual specificity mitogen-activated protein kinase kinase 2 | |
| (MAP kinase kinase 2; MAPKK 2); ERK activator kinase 2; | |
| MAPK/ERK kinase 2 (MEK2) | L11285 |
| dual specificity mitogen-activated protein kinase kinase 5 | |
| (MAP kinase kinase 5; MAPKK 5) | U25265 |
| ribosomal protein S6 kinase II alpha 1 (S6KII-alpha 1); | L07597 |
| ribosomal S6 kinase 1 (RSK1) | |
| B-lymphocyte germinal center kinase (GC kinase) | U07349 |
| YSK1; Ste20 & SPS1-related kinase | D63780 |
| protein phosphatase 2B regulatory subunit; | M30773 |
| calcineurin B subunit isoform 1 | |
| protein-tyrosine phosphatase MEG2 (PTPASE-MEG2) | M83738 |
| protein-tyrosine phosphatase alpha precursor | M34668 |
| (R-PTP-alpha; PTPRA; PTPA) | |
| ras associated with diabetes (RAD1) | L24564 |
| CDC42 homolog; G25K GTP-binding protein | |
| (brain isoform + placental isoform) | M35543 + M57298 |
| calmegin | D86322 |
| calbindin; avian-type vitamin D-dependent calcium binding protein (CABP); D-28K | X06661 |
| stratifin (SFN); 14-3-3 protein sigma; epithelial cell marker protein 1; HME1 | AF029082 |
| FKBP-rapamycin associated protein (FRAP); rapamycin target protein | L34075 |
| zinc finger protein 37 (ZFP37); KRAB domain zinc finger protein | AF022158 |
| CCAAT/enhancer-binding protein epsilon (C/EBP epsilon; CEBPE) | U48866; U48865 |
| transcription initiation factor IID; TATA-box factor; | |
| TATA sequence-binding protein (TBP) | M34960 |
| 60S ribosomal protein L6 (RPL6); | (TAXREB107); |
| TAX-responsive enhancer element binding protein 107 | |
| neoplasm-related protein C140 | X69391 |
| DNA-binding protein HIP116; ATPase; SNF2/SWI2-related protein | L34673 |
| basic transcription factor 2 44-kDa subunit (BTF2p44) | Z30094 |
| octamer-binding transcription factor 2 (oct-2; OTF2); lymphoid-restricted | |
| immunoglobulin octamer binding protein NF-A2; POU2F2 | M36542 |
| zinc finger protein 40 (ZNF40); human immunodeficiency virus type I | |
| enhancer-binding protein 1 (HIV-EP1); | |
| major histocompatibility complex binding protein 1 (MBP-1); | |
| positive regulatory domain II binding factor 1 (PRDII-BF1) | X51435 |
| nervous-system specific octamer-binding transcription factor N-oct3; | |
| N-oct5A & N-oct5B; brain-specific homeobox/ | |
| POU domain protein 2 (POU3F2); | |
| brn2; oct7 | Z11933 |
| hypoxia-inducible factor 1 alpha (HIF1 alpha); ARNT-interacting protein; | |
| member of PAS protein 1 (MOP1) | U22431 |
| CCAAT/enhancer binding protein alpha (C/EBP alpha) | U34070 |
| HOMEOBOX PROTEIN MOX-2 (GROWTH ARREST-SPECIFIC HOMEOBOX) | X82629 |
| endothelial transcription factor GATA2 | M68891 |
| DNA-binding protein inhibitor Id-2 | M97796 |
| activating transcription factor 4 (ATF4); | |
| tax-responsive enhancer element B67 (TAXREB67); | |
| cAMP-response element-binding protein 2 (CREB2) | D90209 |
| heat shock factor protein 1 (HSF1); heat shock transcription factor 1 (HSTF1); TCF5 | M64673 |
| FK506-binding protein 13 precursor (FKBP13); | |
| FKBP2; peptidyl-prolyl cis-trans isomerase (PPIase) | M65128 |
| cAMP response element binding protein (CRE-BP1); transcription factor ATF2; HB16 | M31630 |
| cAMP-response element binding protein (CREB) | M34356 |
| early growth response protein 1 (EGR1); transcription factor ETR103; KROX24; | |
| zinc finger protein 225 (ZNF225); AT225 | X52541; M62829 |
| tristetraproline (TTP); TIS11; ZFP36; | |
| growth factor-inducible nuclear protein 475 (NUP475) | M92843 |
| purine-rich single-stranded DNA-binding protein alpha (PURA) | M96684 |
| transcription factor relB; I-rel | M83221 |
| CYCLIC-AMP-DEPENDENT TRANSCRIPTION FACTOR ATF-3 | |
| (ACTIVATING FACTOR 3) | L19871 |
| octamer-binding transcription factor 1 (oct-1; OTF1); | |
| octamer binding protein NF-A1; POU2F1 | X13403 |
| B-cell lymphoma 3-encoded protein (bcl-3) | M31732 |
| retinoic acid receptor gamma 1 (RAR-gamma 1; RARG) | M24857; M38258; |
| | M57707; M32074 |
| PRB-binding protein E2F1; retinoblastoma-binding protein 3 (RBBP3); | |
| retinoblastoma-associated protein 1 (RBAP1); PBR3 | M96577 |
| retinoic acid receptor alpha; retinoid X receptor alpha (RXRA) | X52773 |
| major histocompatibility complex enhancer-binding protein MAD3 | M69043 |
| fuse-binding protein 2 (FBP2) | U69126 |
| methyl CpG-binding protein 2 (MECP2) | L37298 |
| AP4 basic helix-loop-helix DNA-binding protein | S73885 |
| hepatocyte nuclear factor 4 (HNF4); transcription factor 14 | X76930 |
| metal-regulatory transcription factor | X78710 |
| cockayne syndrome group A; WD-repeat protein (CSA protein) | U28413 |
| RNase L inhibitor | X76388 |
| 40S ribosomal protein S5 | U14970 |
| glutamic-pyruvate transaminase 1 (GPT1); | D10355 |
| alanine aminotransferase 1 (AAT1) | |
| peptidylprolyl cis-trans isomerase A (PPIase; PPIA); | |
| rotamase; cyclophilin A (CYPA); | |
| cyclosporin A-binding protein | Y00052 |
| probable protein disulfide isomerase ER-60 precursor | |
| (ERP60); 58-kDa microsomal protein; | |
| phospholipase C alpha | D16234; Z49835; |
| | D83485; U42068 |
| HSC70-interacting protein; progesterone receptor-associated P48 protein | U28918 |
| chaperonin-containing T-complex polypeptide 1 beta subunit | |
| (CCT-beta; CCTB; CCT2; TCP1-beta); 99D8.1 | AF026293 |
| peroxisome assembly factor-2 (PAF-2); | |
| peroxisomal-type ATPase 1; peroxin-6; PEX6; | |
| PXAAA1 | U56602 |
| CELLULAR RETINOIC ACID BINDING PROTEIN | S74445 |
| endothelin-converting enzyme 1 | Z35307 |
| matrix metalloproteinase 14 precursor (MMP14); MMP-X1; | |
| membrane-type matrix metalloproteinase 1 (MT- | |
| MMP1) | D26512; X83535 |
| bleomycin hydrolase (BLM hydrolase) | X92106 |
| proteasome activator HPA28 subunit beta | D45248 |
| placental plasminogen activator inhibitor 2 | |
| (PAI-2; PLANH2); monocyte ARG-serpin; | |
| urokinase inhibitor | M18082; J02685 |
| alpha-2-macroglobulin precursor (alpha-2-M) | M11313 |
| tissue inhibitor of metalloproteinase 1 precursor (TIMP1); | |
| erythroid potentiating activity (EPA); fibroblast collagenase inhibitor | X03124 |
| alpha-1-antichymotrypsin precursor (ACT) | K01500 |
| alpha-1-antitrypsin precursor; alpha-1 protease inhibitor; alpha-1-antiproteinase | X02920 |
| DNA-binding protein A (DBPA); cold shock domain protein A (CSDA) | M24069 |
| decoy receptor 3 (DCR3) | AF104419 |
| T-complex protein 1 zeta-like subunit (CCT-zeta-like; TCP1-zeta-like); TSA303; | |
| testis-specific TCP20# | D78333 |
| chromatin assembly factor 1 p48 subunit (CAF1 p48 subunit); | |
| retinoblastoma-binding protein 4 (RBBP4); RBAP48; msil protein homolog | X74262 |
| high mobility group protein HMG2 | X62534 |
| DNA-binding protein UEV-1; UBE2V | U49278 |
| activator 1 140-kDa subunit (A1 140-kDa subunit); | |
| replication factor C large subunit; | |
| DNA-binding protein PO-GA | L14922 |
| replication factor C 36-kDa subunit (RFC36); activator 1 36-kDa subunit | L07540 |
| replication factor C 38-kDa subunit (RFC38); activator 1 38-kDa subunit | L07541 |
| replication protein A 70-kDa subunit (RPA70; REPA1; RF-A); | |
| single-stranded DNA-binding protein | M63488 |
| activator 1 40-kDa subunit (A1 40-kDa subunit); | |
| replication factor C 40-kDa subunit (RFC40); RFC2 | M87338 |
| activator 1 37-kDa subunit; replication factor C 37-kDa subunit (RFC37); RFC4 | M87339 |
| DNA topoisomerase 1 (TOP1) | J03250 |
| DNA topoisomerase II alpha (TOP2A) | J04088 |
| proliferating cyclic nuclear antigen (PCNA); cyclin | M15796; |
| | J04718 |
| DNA topoisomerase II beta (TOP2B) | X68060 |
| replication protein A 14-kDa subunit (RP-A) (RF-A); replication factor A protein 3 | L07493 |
| DNA nucleotidylexotransferase; terminal addition enzyme; | |
| terminal deoxynucleotidyltransferase (TDT); terminal transferase; DNTT | M11722; K01919 |
| DNA polymerase delta catalytic subunit | M80397 |
| DNA topoisomerase III (TOP3) | U43431 |
| excision repair cross-complementing rodent repair deficiency | |
| complementation group 6 (ERCC6); | |
| Cockayne syndrome protein 2 type B (CSB) | L04791 |
| xeroderma pigmentosum group G complementing protein (XPG); X-ray repair- | |
| complementing defective repair in Chinese hamster cells 5 (XRCC5) | L20046; X69978 |
| Ku (p70/p80) subunit; ATP-dependent DNA helicase II 86-kDa subunit; | |
| lupus ku autoantigen protein; thyroid-lupus autoantigen (TLAA); | |
| CTC box binding factor 85-kDa subunit (CTCBF; CTC85); nuclear factor IV | M30938 |
| xeroderma pigmentosum group B complementing protein (XPB); | |
| excision repair cross-complementing rodent | |
| repair deficiency complementation group 3 (ERCC3); | |
| basal transcription factor 2 89-kDa subunit (BTF2-p89; TFIIH 89-kDa subunit) | M31899 |
| Ku 70-kDa subunit; ATP-dependent DNA helicase II 70-kDa subunit; | |
| lupus ku autoantigen protein P70; thyroid-lupus auto-antigen (TLAA); | |
| CTC box binding factor 75-kDa subunit (CTC75) | M32865; S38729 |
| X-ray repair-complementing defective repair in Chinese hamster cells 1 (XRCC1) | M36089 |
| ubiquitin-conjugating enzyme E2 17-kDa (UBE2A); ubiquitin-protein ligase; | |
| ubiquitin carrier protein; HR6A | M74524 |
| DNA polymerase alpha catalytic subunit (POLA) | X06745 |
| 6-O-methylguanine-DNA methyltransferase (MGMT); methylated-DNA-protein- | |
| cysteine methyltransferase | M29971 |
| xeroderma pigmentosum group D complementing protein (XPD); X-ray repair- | |
| complementing defective repair in Chinese hamster cells 2 (XRCC2) | X52221 |
| excision repair cross-complementing rodent repair deficiency | |
| complementation group 1 (ERCC1) | M13194 |
| mutL protein homolog1 (MLH1); colon cancer nonpolyposis type 2 protein (COCA2) | U07418 |
| UV excision repair protein RAD23 homolog B (HHR23B); | |
| xeroderma pigmentosum group C repair complementing complex 58-kDa protein | D21090 |
| HHR23A; UV excision repair protein protein RAD23A | D21235 |
| DNA-dependent protein kinase (DNA-PK) + | |
| DNA-PK catalytic subunit (DNA-PKCS) | U35835 + U47077 |
| DNA damage repair & recombination protein 52 (RAD52) | U12134 |
| ataxia telangiectasia (ATM) | U33841 |
| RAD50 | U63139 |
| DNA ligase IV (LIG4); polydeoxyribonucleotide synthase | X83441 |
| DNA ligase III (LIG3); polydeoxyribonucleotide synthase | X84740 |
| DNA mismatch repair protein MSH2 | U04045; L47583 |
| DNA mismatch repair protein MSH6; mutS alpha 160-kDa subunit; | |
| G/T mismatch binding protein (GTMBP; GTBP) | U54777 |
| RecQ protein-like (DNA helicase Q1-like) | D37984 |
| DNA polymerase beta subunit (DPOB) | D29013 |
| DNA mismatch repair protein PMS1 (PMS1 protein homolog 1) | U13695 |
| DNA mismatch repair protein PMS2 (PMS1 protein homolog 2) | U13696 |
| ATP-dependent DNA ligase I (LIG1); polydeoxyribonucleotide synthase | M36067 |
| xeroderma pigmentosum group A complementing protein (XPA) | D14533 |
| damage-specific DNA binding protein p48 subunit (DDBB P48); implicated in xeroderma | |
| pigmentosum group E (DDB2) | U18300 |
| DNA repair protein XRCC4 | U40622 |
| G/T mismatch-specific thymine DNA glycosylase (TDG) | U51166 |
| DNA repair protein XRCC9 | U70310 |
| endonuclease III homolog 1; HNTH1; OCTS3 | U79718 |
| DNA-repair protein complementing XP-C cells; xeroderma pigmentosum group C | |
| complementing protein (p125) | D21089 |
| uracil-DNA glycosylase precursor (UNG1) | X15653 |
| DNA-(apurinic or apyrimidinic site) lyase; AP endonuclease 1 (APE1); | |
| apurinic/apyrimidinic endonuclease (APEX); APEX nuclease (APEN); | |
| REF1 | X59764; X66133 |
| DNA repair protein RAD54 homolog | X97795 |
| recA-like protein HsRad51; DNA repair protein RAD51 homolog | D13804 |
| V(D)J recombination activating protein 2 (RAG2) | M94633 |
| V(D)J recombination activating protein 1 (RAG1) | M29474 |
| muscle-specific DNase I-like precursor (DNase1L1; DNL1L); DNase X | X90392; L40817; U06846 |
| deoxyribonuclease I (DNase I) | M55983 |
| dual-specificity protein phosphatase 9; | |
| mitogen-activated protein kinase phosphatase 4 | |
| (MAP kinase phosphatase 4 (MKP4) | Y08302 |
| G1/S-specific cyclin D3 (CCND3) | M92287 |
| G1/S-specific cyclin D1 (CCND1); cyclin parathyroid adenomatosis 1 (PRAD1); | |
| bcl-1 oncogene | X59798 |
| G1/S-specific cyclin D2 (CCND2) + KIAK0002 | M90813 + D13639 |
| G2/mitotic-specific cyclin B1 (CCNB1) | M25753 |
| G1/S-specific cyclin E (CCNE) | M73812 |
| G2/mitotic-specific cyclin G1 (CCNG1; CYCG1) | U47413 |
| G1/S-specific cyclin C | M74091 |
| cyclin K | AF060515 |
| protein serine/threonine kinase STK1; cell division protein kinase 7 (CDK7); | |
| CDK-activating kinase (CAK); 39-kDa protein kinase | L20320 |
| cyclin-dependent protein kinase 2 (CDK2); p33 protein kinase | M68520 |
| extracellular signal-regulated kinase 2 (ERK2); | |
| mitogen-activated protein kinase 2 (MAP kinase 2; MAPK 2); p42-MAPK | M84489 |
| mitogen-activated protein kinase 3 (MAPK3; PRKM3); MAPK1; | |
| extracellular signal-regulated kinase 1 (ERK1); | |
| microtubule-associated protein 2 kinase; insulin-stimulated MAP2 kinase | X60188 |
| extracellular signal-regulated kinase 3 (ERK3); MAP kinase 3 (MAPK3; p97-MAPK); | |
| PRKM5 | X80692 |
| CDC-like kinase 3 (CLK3) | L29220 |
| cell division protein kinase 4; cyclin-dependent kinase 4 (CDK4); PSK-J3 | M14505 |
| extracellular signal-regulated kinase 5 (ERK5); BMK1 kinase | U25278 |
| cell division control protein 2 homolog (CDC2); p34 protein kinase; | |
| cyclin-dependent kinase 1 (CDK1) | X05360 |
| extracellular signal-regulated kinase 4 (ERK4); MAP kinase 4 (MAPK4; p63-MAPK); | |
| PRKM4 | X59727 |
| cell division protein kinase 5 (CDK5); tau protein kinase II catalytic subunit | |
| (TPKII catalytic subunit); serine/threonine protein kinase PSSALRE | X66364 |
| extracellular signal-regulated kinase 6 (ERK6); stress-activated protein kinase-3; | |
| mitogen-activated protein kinase p38 gamma; (MAP kinase p38 gamma) | X79483 |
| serine/threonine-protein kinase PLK1 (STPK13) | U01038 |
| checkpoint kinase 1 (CHK1) | AF016582 |
| aurora- & IPL1-like midbody-associated protein | |
| kinase 1 (AIM1); ARK2 | AF008552 |
| cyclin G-associated kinase (GAK) | D88435 |
| special AT-rich sequence binding protein 1 | M97287 |
| (SATB1); MAR/SAR DNA-binding protein | |
| cyclin-dependent kinase inhibitor 1A (CDKN1A); | |
| melanoma differentiation-associated protein 6 (MDA6); | |
| CDK-interacting protein 1 (CIP1); WAF1; SDI1 | U09579; L25610 |
| wee1Hu CDK tyrosine 15-kinase; wee-1-like protein kinase | U10564 |
| cyclin-dependent kinase 4 inhibitor 2B (CDKN2B); p14-INK4B; | |
| multiple tumor suppressor 2 (MTS2) | U17075; L36844 |
| helix-loop-helix protein HLH 1R21; DNA-binding protein inhibitor Id-3; HEIR-1 | X69111 |
| DNA-binding protein inhibitor ID-1; Id-1H | D13889 |
| prothymosin alpha (PROT-alpha; PTMA) | M26708 |
| 40S ribosomal protein S19 (RPS19) | M81757 |
| p55CDC | U05340 |
| cell division cycle protein 25A (CDC25A); M-phase inducer phosphatase 1 | M81933 |
| CDC25B; CDC25HU2; M-phase inducer phosphatase 2 | M81934; S78187 |
| CDC25C; M-phase inducer phosphatase 3 | M34065 |
| growth inhibitory factor (GIF); metallothionein-III (MT-III; MT3) | D13365; |
| | M93311 |
| CDC37 homolog | U63131 |
| cell cycle protein P38-2G4 homolog; HG4-1 | U59435 |
| btg protein precursor; NGF-inducible anti-proliferative protein PC3 | U72649 |
| RCL growth-related c-myc-responsive gene | AF040105 |
| 40-kDa heat-shock protein 1 (HSP40); DNAJ protein homolog 1 (HDJ1; DNAJ1) | D49547 |
| 60-kDa heat shock protein (HSP60); HSPD1; 60-kDa chaperonin; | |
| mitochondrial matrix protein P1 precursor; p60 lymphocyte protein; | |
| HUCHA60; GROEL | M34664 |
| 90-kDa heat-shock protein A (HSP90A); HSP86; HSPCA | X07270 |
| 27-kDa heat-shock protein (HSP27); stress-responsive protein 27 (SRP27); | |
| estrogen-regulated 24-kDa protein; HSPB1 | X54079 |
| 70-kDa heat shock protein 1 (HSP70.1; HSPA1) | M11717 |
| heat shock 70-kDa protein 6 (heat shock 70-kDa protein B) | X51757; M11236 |
| heat shock cognate 71-kDa protein; heat shock 70-kDa protein 8 (HSPA8; HSC70; | |
| HSP73 | Y00371 |
| heat shock-related 70-kDa protein 2 | L26336 |
| major vault protein (MVP); lung resistance-related protein (LRP) | X79882 |
| thiosulfate sulfurtransferase; rhodanese | D87292 |
| soluble epoxide hydrolase (SEH); epoxide hydratase; cytosolic epoxide hydrolase (CEH); | |
| EPHX2 | L05779 |
| serum paraoxonase/arylesterase 1 (PON1); serum aryldiakylphosphatase 1; | |
| aromatic esterase 1 (A-esterase 1) | M63012 |
| polymorphic arylamine N-acetyltransferase (PNAT) + monomorphic (MNAT) | X14672; X17059 |
| quinone oxidoreductase; NADPH: quinone reductase; zeta-crystallin (CRYZ) | L13278; S58039 |
| cytosolic superoxide dismutase 1 (SOD1) | K00065; X02317 |
| cytochrome P450 IB1 (CYP1B1) | U03688 |
| cytochrome P450 IIA6 (CYP2A6) + CYP2A7 + CYP2A13 + | M33318; M33316 |
| CYP2A7PT + CYP2A7PC + | |
| U22029 + U22030 + U22044 | |
| cytochrome P450 IIB6 (CYP2B6) + CYP2B3 | M29874; J02864 |
| cytochrome P450 IIIA3 (CYP3A3) + CYP3A4 + CYP3A5 + CYP3A7 | |
| M13785 + M18907 + J04813 + D00408 | |
| cytochrome P450 IVA11 (CYP4A11) | L04751 |
| cytochrome P450 VIIA1 (CYP7A1) | X56088 |
| D-amino acid oxidase (DAMOX; DAO; DAAO) | X13227 |
| S-mephenytoin 4 hydroxylase; cytochrome P450 IIC9 (CYP2C9) + CYP2C10 + | |
| CYP2C17 + CYP2C18 + CYP2C19 | M21940 + M15331; |
| | M21939 + M61858 + |
| | M61854 |
| cytochrome P450 IIE1 (CYP2E1) | J02625 |
| cytochrome P450 IIF1 (CYP2F1) | J02906 |
| cytochrome P450 IVB1 (EC 1.14.14.1) (P450-HP) | J02871 |
| cytochrome P450 IA2 (P450-P3) (P450-4) | Z00036 |
| plasma glutathione peroxidase precursor (GPXP; GPX3) | D00632; X58295 |
| natural killer cell enhancing factor (NKEFB) + thiol-specific antioxidant protein | |
| (TSA); thioredoxin peroxidase 1 (TDPX1); | |
| thioredoxin-dependent peroxide reductase 1 | L19185 + Z22548; |
| | X82321 |
| thioredoxin peroxidase 2 (TDPX2); thioredoxin-dependent peroxide reductase 2; | |
| proliferation-associated gene (PAG); | |
| natural killer cell enhancing factor A (NKEFA) | X67951 |
| glutathione reductase (GRase; GSR; GR) | X15722 |
| microsomal glutathione S-transferase 12 (GST12; MGST1) | J03746; B28083 |
| glutathione S-transferase pi (GSTP1; GST3) | X08058; M24485 |
| glutathione peroxidase (GSHPX1; GPX1) | Y00483; M21304 |
| glutathione S-transferase theta 1 (GSTT1) | X79389 |
| methallothionein IH (MT1H); metallothinein0 | |
| (MT0) + MT1I; MT2 + MT1L + MT1R | X64177 + X97260 + |
| | X76717 + X97261 |
| glutathione peroxidase-gastrointestinal (GSHPX-GI); | |
| glutathione peroxidase-related protein 2 (GPRP) | X53463 |
| heme oxygenase 1 (HO1); HSOXYGR | X06985 |
| heme oxygenase 2 (HO2) | D21243; S34389 |
| dimethylaniline monooxygenase (N-oxide forming) 1 (EC 1.14.13.8); | |
| fetal hepatic flavin-containing monooxygenase 1 (FMO 1); | |
| dimethylaniline oxidase 1 | M64082 |
| glutathione S-transferase mu1 (GSTM1; GST1); HB subunit 4; GTH4 | X68676; S01719 |
| glutathione S-transferase A1 (GTH1; GSTA1); HA subunit 1; GST-epsilon | M25627 |
| glutathione-S-transferase (GST) homolog | U90313 |
| glutathione synthetase (GSH synthetase; GSH-S); glutathione synthase | U34683 |
| NAD(P)H dehydrogenase; quinone reductase; DT-diaphorase; | |
| azoreductase; phylloquinone reductase; menadione reductase | J03934 |
| growth arrest & DNA-damage-inducible protein (GADD45); | |
| DNA-damage-inducible transcript 1 (DDIT1) | M60974 |
| tumor necrosis factor alpha precursor (TNF-alpha; TNFA); cachectin | X01394 |
| lymphotoxin-alpha precursor (LT-alpha); tumor necrosis factor-beta (TNF-beta; TNFB) | D12614 |
| fas antigen ligand (FASL); apoptosis antigen ligand (APTL; APT1LG1); | |
| TNFSF6 | D38122; |
| U08137 | |
| tumor necrosis factor receptor (TNFR) + tumor necrosis factor receptor 2 (TNFR2); | |
| tumor necrosis factor binding protein 2 (TBP2) | M32315 + M55994 |
| tumor necrosis factor receptor 1 (TNFR1); tumor necrosis factor binding protein 1 | |
| (TBP1); CD120A antigen | M33294 |
| fasL receptor; apoptosis-mediating surface antigen fas; APO-1 antigen; CD95 antigen | M67454 |
| retinoic acid receptor beta (RXR-beta; RXRB) | M84820; X63522; S54072 |
| tumor necrosis factor receptor 1-associated death domain protein | |
| (TNFR1-associated death domain protein; TRADD) | L41690 |
| CD27BP (Siva) | U82938 |
| tumor necrosis factor type 1 receptor associated protein (TRAP1) | U12595 |
| caspase-2 precursor (CASP2); ICH-1L protease + ICH-1S protease | U13021 + U13022 |
| interleukin-1 beta convertase precursor (IL-1BC); IL-1 beta converting enzyme (ICE); | |
| p45; caspase-1 (CASP1) | U13699; M87507; X65019 |
| caspase-6 precursor (CASP6); cysteine protease MCH2 isoforms alpha + beta | U20536 + U20537 |
| caspase-4 precursor (CASP4); ICH-2 protease; TX protease; | |
| ICE(REL)-II + caspase-5 precursor (CASP5); ICH-3 protease; TY protease; | |
| ICE(REL)-III | U28014 + U28015 |
| caspase-7 precursor (CASP7); ICE-like apoptotic protease 3 (ICE-LAP3); | |
| apoptotic protease MCH-3; CMH-1 | U37448 |
| TNF-related apoptosis inducing ligand (TRAIL); APO-2 ligand (APO2L) | U57059 |
| caspase-8 precursor (CASP8); ICE-like apoptotic protease 5 (ICE-LAP5); | |
| MORT1-associated CED-3 homolog (MACH); | |
| FADD-homologous ICE/CED-3-like protease (FADD-like ICE; FLICE); | |
| apoptotic cysteine protease MCH-5 | U60520; U58143; |
| | X98172; AF00962 |
| apoptosis regulator bax | L22474 |
| apoptosis regulator bcl-x | Z23115; L20121; L20122 |
| apoptosis regulator bcl-2 | M14745 |
| NIP3 (NIP3) | U15174 |
| bcl2 homologous antagonist/killer (BAK) | U23765; U16811; X84213 |
| induced myeloid leukemia cell differentiation protein MCL-1 | L08246 |
| BAD protein; bcl-2 binding component 6 (BBC6); bcl-2L8 | U66879 |
| BCL-2 binding athanogene-1 (BAG-1); | |
| glucocorticoid receptor-associated protein RAP46 | S83171; Z35491 |
| interferon-inducible RNA-dependent protein kinase (P68 kinase) | M35663; U50648 |
| inducible nitric oxide synthase (INOS); type II NOS; hepatocyte NOS (HEP-NOS) | L09210 |
| defender against cell death 1 (DAD1) | D15057 |
| clusterin precursor (CLU); complement-associated protein SP-40; | |
| complement lysis inhibitor (CLI); apolipoprotein J (APOJ); | |
| testosterone-repressed prostate message 2 (TRPM2); | |
| sulfated glycoprotein 2 (SGP2) | M74816 |
| growth arrest & DNA-damage-inducible protein 153 (GADD153); | |
| DNA-damage-inducible transcript 3 (DDIT3); | |
| C/EBP homologous protein (CHOP) | S40706; S62138 |
| inhibitor of apoptosis protein1 (HIAP1; API1) + IAP homolog C; | |
| TNFR2-TRAF signaling complex protein 1; MIHC | U45878 + U37546 |
| cytoplasmic dynein light chain 1 (HDLC1); | |
| protein inihibitor of neuronal nitric oxide synthase (PIN) | U32944 |
| apoptosis inhibitor survivin | U75285 |
| sentrin; ubiquitin-like protein SMT3C; | |
| ubiquitin-homology domain protein PIC1; | |
| UBL1; SUMO-1; GAP modifying protein 1; GMP1 | U83117 |
| IEX-1L anti-death protein; PRG-1; DIF-2 | AF039067; AF071596 |
| poly(ADP-ribose) polymerase (PARP; PPOL); ADPRT; | |
| NAD + ADP-ribosyltransferase; poly(ADP-ribose) synthetase | M18112; J03473 |
| avian myelocytomatosis viral oncogene homolog (MYC) | V00568 |
| p53-associated mdm2 protein | Z12020; M92424 |
| platelet-derived growth factor B subunit precursor (PDGFB; PDGF2); | |
| bacaplermin; c-sis | X02811; X02744; |
| | M12783; M16288 |
| p53 cellular tumor antigen | M14694; |
| M14695 | |
| MYB-related protein B (B-MYB); avian myeloblastosis viral | |
| oncogene homolog-like 2 (MYBL2) | X13293 |
| triiodothyronine receptor; thyroid hormone receptor (THRA1); | |
| v-erbA-related protein ear-1 | M24898 |
| jun proto-oncogene; avian sarcoma virus 17 oncogene homolog; | |
| transcription factor AP-1 | J04111 |
| insulin-like growth factor binding protein 2 (IGFBP2) | M35410 |
| c-myc purine-binding transcription factor puf; nucleoside diphosphate kinase B | |
| (NDP kinase B; NDKB) + nm23-H2S | L16785 + M36981 |
| Abelson murine leukemia viral oncogene homolog 1 (ABL1) | M14752 |
| retinoblastoma-associated protein (RB1); PP110; P105-RB | M15400 |
| L-myc proto-oncogene (MYCL1) | M19720 |
| breast cancer type 2 susceptibility protein (BRCA2) | U43746 |
| fos-related antigen (FRA1); fosL1 | X16707 |
| nucleolar phosphoprotein B23; nucleophosmin (NPM); numatrin | M23613 |
| c-myc binding protein MM-1 | D89667 |
| c-fos proto-oncogene; G0S7 protein | K00650 |
| met proto-oncogene; hepatocyte growth factor | J02958 |
| receptor precursor (HGF-SF receptor) | |
| nucleoside diphosphate kinase A (NDKA); NDP kinase A; | |
| tumor metastatic process-associated protein; | |
| metastasis inhibition factor NM23 (NM23-H1) | X17620 |
| matrix metalloproteinase 11 (MMP11); stromelysin 3 | X57766 |
| box-dependent myc-interacting protein 1 | U68485 |
| H-ras proto-oncogene; transforming G protein | V00574 |
| protein-tyrosine phosphatase PTEN; | |
| mutated in multiple advanced cancers 1 (MMAC1); | |
| TEP1 | U92436 |
| prostaglandin G/H synthase 2 precursor (PGH synthase 2; PGHS2; PTGS2); | |
| cyclooxygenase 2 (COX2); prostaglandin-endoperoxide synthase 2 | M90100 |
| 78-kDa glucose regulated protein precursor (GRP 78); | |
| immunoglobulin heavy chain binding protein (BIP) | M19645 |
| complement 3 (C3) | K02765 |
| interleukin-10 precursor (IL-10); cytokine synthesis inhibitory factor (CSIF) | M57627 |
| thioredoxin (TRDX; TXN); ATL-derived factor (ADF); | |
| surface-associated sulphydrylprotein (SASP) | J04026 |
| enolase 1 alpha (ENO1); non-neural enolase (NNE); | M14328 |
| phosphopyruvate hydratase (PPH) | |
| biliverdin reductase A precursor (BLVRA; BVR) | U34877 |
| tyrosine aminotransferase (TAT); I-tyrosine: 2-oxoglutarateaminotransferase | X52520 |
| muscle-specific carbonic anhydrase III (CA3); carbonate dehydratase III | M29458 |
| spermidine/spermine N1-acetyltransferase (SSAT); | |
| diamine acetyltransferase; putrescine acetyltransferase | M55580 |
| L-lactate dehydrogenase H subunit (LDHB) | Y00711 |
| phosphoglyceride kinase 1 (PGK1; PGKA); primer recognition protein 2 (PRP2) | V00572 |
| glucose-6-phosphate dehydrogenase (G6PD) | X03674 |
| mitochondrial phosphoenolpyruvate carboxykinase 2 precursor (PEPCK-M; PCK2); | |
| phosphoenolpyruvate carboxylase | X92720 |
| galactoside 2-1-fucosyltransferase 2; | |
| GDP-1-fucose: beta-D-galactoside 2-alpha-1-fucosyltransferase 2; | |
| fucosyltransferase 2 (FUT2); secretor blood group alpha-2-fucosyltransferase; | |
| secretor factor 2 (SE2) | D87942 |
| galactosyltransferase-associated protein kinase p58 (GTA); | |
| cell division cycle 2-like 1 (CDC2L1; CLK1) | M37712 |
| adrenodoxin | M34788 |
| alcohol dehydrogenase alpha subunit + alcohol | |
| dehydrogenase 2 + alcohol | |
| dehydrogenase 3 | M12271 + D00137 + X04299 |
| alcohol dehydrogenase 5 chi polypeptide | M30471 |
| alcohol dehydrogenase class II pi subunit | M15943 |
| CREATINE KINASE B CHAIN | L47647 |
| fatty acid synthase | S80437 |
| hepatic triglyceride lipase (HTGL) | X07228 |
| bile-salt-activated lipase | M85201; M37044 |
| mitochondrial enoyl-CoA hydratase short subunit 1 | D13900 |
| peroxisomal bifunctonal enzyme | L07077 |
| peroxisomal acyl-CoA oxidase branched subunit (BRCOX) | X95190 |
| acyl-CoA dehydrogenase long chain-specific precursor (LCAD; ACADL) | M74096 |
| alcohol sulfotransferase | L20000 |
| estradiol 17 beta-dehydrogenase 1 | M36263 |
| cytochrome P450 XVIIA1 (CYP17A1) | M14564 |
| peroxisomal 3-ketoacyl-CoA thiolase precursor (PTHIO); | |
| peroxisomal 3-oxoacyl-CoA thiolase; beta-ketothiolase; | |
| acetyl-CoA acyltransferase (ACAA) | X14813 |
| 3-hydroxy-3-methylglutaryl-coenzyme A reductase (HMG-CoA reductase; HMGCR) | M11058 |
| lipoprotein lipase precursor (LPL) | M15856 |
| lung group IB phospholipase A2 precursor (PLA2); | |
| phosphatidylcholine 2-acylhydrolase | M21054 |
| mitochondrial cytochrome P450 XIA1 precursor; P450(SCC); | |
| cholesterol side-chain cleavage enzyme; cholesterol desmolase CYP11A1 | M14565 |
| dihydrofolate reductase (DHFR) | V00507 |
| thymidylate synthase (TYMS; TS) | X02308 |
| cytosolic thymidine kinase (TK1) | K02581 |
| ribonucleoside-diphosphate reductase M1 subunit; ribonucleotide reductase | X59543 |
| microsomal UDP-glucuronosyltransferase 2B15 precursor (UDPGT); UDPGTH-3; | |
| UGT2B15 + microsomal 2B10 precursor (UDPGT); UGT2B10 + 2microsomal | |
| B8 precursor | U08854; X63359; |
| | U06641; J05428; |
| | Y00317 |
| GLCLC, GLCL (Glutamate-cysteine ligase catalytic subunit, | |
| gamma-glutamylcysteine synthetase) | M90656 |
| gamma-glutamyl hydrolase precursor (GGH; GH); | |
| folylpolygammaglutamyl hydrolase; | |
| gamma-glu-X carboxypeptidase; conjugase | U55206 |
| 3′-phosphoadenosine 5′-phosphosulfate | |
| synthase 1 (PAPS synthase 1; PAPSS1); | |
| PAPS synthetase 1; sulfurylase kinase 1 (SK1) | Y10387 |
| soluble glutamic oxaloacetic transaminase 1 (GOT1); | |
| cytoplasmic aspartate aminotransferase 1; transaminase A | M37400 |
| alcohol dehydrogenase 6 + aldehyde dehydrogenase 1 (ALDH1) | K03000 |
| peroxisomal acyl-coenzyme A oxidase | S69189 |
| very-long-chain-specific acyl-CoA dehydrogenase precursor (VLCAD) | D43682 |
| glutamate-cysteine ligase regulatory subunit (GLCLR); | |
| gamma-glutamylcysteine synthetase | P48507 |
| LOX (Protein-lysine 6-oxidase, Lysyl-Oxidase) | M94054 |
| ornithine decarboxylase | X16277 |
| corticosteroid 11-beta-dehydrogenase isozyme 2 | U14631 |
| cytochrome P450 VA1 (CYP5A1) | M80647 |
| mitochondrial aldehyde dehydrogenase precursor (class 2); ALDHI; ALDH2 | Y00109 |
| 5,6-dihydroxyindole-2-carboxylic acid oxidase precursor (DHICA oxidase); | |
| tyrosinase-related protein 1 (TRP-1); catalase B; glycoprotein-75 (GP75) | X51420 |
| tenascin precursor (TN); hexabrachion (HXB); cytotactin; neuronectin; GMEM; | |
| miotendinous antigen; glioma-associated extracellular matrix antigen | X78565; M55618 |
| OSTEOPONTIN PRECURSOR (BONE SIALOPROTEIN 1) | X13694 |
| ATP-binding cassette transporter (ABCR) | U88667 |
| tissue inhibitor of metalloproteinase 2 precursor (TIMP2) | J05593 |
| matrix metalloproteinase 15 (MMP15) | Z48482 |
| matrix metalloproteinase 14 (MMP14) | D26512 |
| matrix metalloproteinase 1 (MMP1) | X54925 |
| vinculin | M33308 |
| vimentin (VIM) | X56134; M14144 |
| serum amyloid A1 precursor (SAA1) | M23698 |
| senescence marker protein 30 (SMP30); regucalcin (RGN; RC) | D31815 |
| ubiquitin cross-reactive protein precursor (UCRP); | |
| alpha-inducible interferon; interferon-induced 17-kDa protein; G1P2; ISG15 | M13755 |
| laminin gamma 2 subunit precursor (LAMC2) | Z15009 |
| peroxisome assembly factor 1 (PAF1); | |
| peroxisomal membrane protein 3 (PXMP3; PMP3); | |
| 35-kDa peroxisomal membrane protein (PMP35); peroxin 2 (PEX2) | M86852 |
| peroxisomal membrane protein 69 (PMP69) | AF009746 |
| peroxisome biogenesis disorder protein 1 (PEX1) | AF026086 |
| mitochondrial glutamic oxaloacetic transaminase 2 (GOT2); | |
| aspartate aminotransferase 2; transaminase A | M22632 |
| nck, ash & phoshpholipase C gamma-binding protein (NAP4) | AB005216 |
| N-oxide-forming dimethylaniline monooxygenase 4; | |
| hepatic flavin-containing monooxygenase 4 (FMO4) | Z11737 |
| xeroderma pigmentosum group F complementing protein (XPF); | |
| DNA excision repair protein ERCC4; ERCC11 | L77890 |
| replication protein A 30-kDa subunit; replication factor A protein 4 (RPA4; RFA) | U24186 |
| mutY homolog (hMYH) | U63329 |
| beta crystallin A4 (CRYBA4) | U59057 |
| T-complex protein 1 epsilon subunit (TCP1-epsilon); CCT-epsilon (CCTE; CCT5) | D43950 |
| beta crystallin B1 (CRYBB1) | U35340 |
| beta crystallin B2 (CRYBB2); BP | L10035 |
| beta crystallin B3 (CRYBB3; CRYB3) | U71216 |
| mitochondrial 10-kDa heat shock protein (HSP10); | |
| 10-kDa chaperonin (CPN10); HSPE1 | U07550 |
| heat shock protein beta 3 (HSPB3); heat shock 17-kDa protein; HSPL27 | U15590 |
| probable protein disulfide isomerase P5 precursor | D49489 |
| 90-kDa heat-shock protein beta (HSP90); 84-kDa heat-shock protein beta (HSP84); | |
| HSPCB | M16660 |
| microsomal UDP-glucuronosyltransferase 1-6 precursor | |
| (UDPGT; UGT1.6; UGT1F; GNT1) | J04093 |
| glutathione S-transferase mu 3 (GSTM3); GST5 | J05459 |
| cytochrome P450 1A1 (CYP1A1); P450-P1; P450 form 6; P450-C | K03191 |
| peroxisome proliferator activated receptor alpha (PPAR-alpha; PPARA) | L02932 |
| protein disulfide isomerase-related protein precursor (PDIR) | D49490 |
| liver carboxylesterase precursor; acyl coenzyme A: cholesterol acyltransferase (ACAT); | |
| monocyte/macrophage serine esterase (hMSE); CES2 | L07765 |
| serum paraoxonase/arylesterase 3 (PON3); serum aryldiakylphosphatase 3; | |
| aromatic esterase 3 (A-esterase 3) | L48516 |
| cytochrome P450 XXIB (CYP21B); steroid 21-hydroxylase; CYP21A2 | M12792; M23280 |
| cytochrome P450 IID6 (CYP2D6); P450-DB1; debrisoquine 4-hydroxylase | M20403 |
| microsomal UDP-glucuronosyltransferase 1-1 precursor | |
| (UDPGT; UGT1.1; UGT1A; GNT1); bilirubin-specific isozyme 1 (hUG-BR1) | M57899 |
| microsomal UDP-glucuronosyltransferase 1-4 precursor | |
| (UDPGT; UGT1.4; UGT1D; GNT1); bilirubin-specific isozyme 2 (hUG-BR2) | M57951 |
| flavin-containing amine oxidase B (MAOB); monoamine oxidase | M69177 |
| eukaryotic peptide chain release factor subunit 1 (ERF1); TB3-1; C11 protein | M75715 |
| microsomal UDP-glucuronosyltransferase 1-3 precursor | |
| (UDPGT; UGT1.3; UGT1C; GNT1) | M84127 |
| structure-specific recognition protein 1 (SSRP1); | |
| recombination signal sequence recognition protein; T160 | M86737 |
| microsomal UDP-glucuronosyltransferase 1-2 precursor | |
| (UDPGT; UGT1.2; UGT1B; GNT1); HLUGP4 | S55985 |
| thiopurine S-methyltransferase (TPMT) | S62904 |
| meiotic recombination protein DMC1/LIM15 homolog | D63882 |
| short/branched chain-specific acyl-CoA dehydrogenase precursor (SBCAD; ACADSB); | |
| 2-methyl branched chain acyl-CoA dehydrogenase (2-MEBCAD) | U12778 |
| cytochrome P450 XIB1 precursor (CYP11B1); steroid 11-beta-hydroxylase (S11BH) | X55764 |
| cytochrome P450 IVA11 (CYP4A11) | X71480 |
| NADH-cytochrome B5 reductase (B5R); DIA1 | Y09501 |
| coproporphyrinogen III oxidase precursor (CPO); coproporphyrinogenase; | |
| coprogen oxidase (COX) | Z28409 |
| 110-kDa heat-shock protein (HSP110); 105-kDa heat-shock protein (HSP105); | |
| KIAA0201 | D86956 |
| gamma crystallin C (CRYGC; CRYG3; | |
| gamma crystallin 2 + gamma crystallin B (CRYGB; CRYG2); | |
| gamma crystallin 1-2 | U66582 + M11971; M11970 |
| heat shock transcription factor 4 (HHSF4) | D87673 |
| extracellular superoxide dismutase precursor (EC-SOD; SOD3) | J02947 |
| DNAJ protein homolog 2 (DNAJ2; hDJ2; HSJ2) | D13388 |
| DNA mismatch repair protein MSH3; divergent upstream protein (DUP); | |
| mismatch repair protein 1 (MRP1) | J04810 |
| protein disulfide isomerase-related protein ERP72 precursor | J05016 |
| replication protein A 32-kDa subunit (RPA32); | |
| replication factor A protein 2 (REPA2; RPA2; RFA) | J05249 |
| multidrug resistance-associated protein 1 (MRP1) | L05628 |
| calnexin precursor (CANX); | |
| major histocompatibility complex class I | |
| antigen-binding protein p88; IP90 | L10284; L18887; |
| | M94859; M98452 |
| cyclophilin-40 (CYP40; CYPD); 40-kDa peptidyl-prolyl cis-trans isomerase (PPIASE); | |
| rotamase; cyclophilin-related protein | L11667 |
| heat shock 70-kDa protein 4 (HSPA4); HSP70RY; | |
| heat shock 70-related protein APG-2 | L12723 |
| T-complex protein 1 theta subunit (TCP1-theta); | |
| CCT-theta (CCTQ; CCT8); KIAA0002 | D13627 |
| mitochondrial stress-70 protein precursor; 75-kDa glucose-regulated protein (GRP75); | |
| peptide-binding protein 74 (PBP74); mortalin (MOT); HSPA9B | L15189 |
| p23; 23-kDa progesterone receptor-associated protein | L24804; L24805 |
| FLAP endonuclease 1 (FEN1); maturation factor 1 (MF1) | L37374 |
| FK506-binding protein 12 (FKBP12); peptidyl-prolyl cis-trans isomerase (PPIase); | |
| rotamase | M34539; M80199; |
| | M92423; X55741; |
| | X52220 |
| heat shock factor protein 2 (HSF2); heat shock transcription factor 2 (HSTF2) | M65217 |
| 3-methyladenine DNA glycosylase (ADPG); 3-alkyladenine DNA glycosylase; | |
| N-methylpurine-DNA glycosirase (MPG) | M74905 |
| calreticulin precursor (CRP55); calregulin; HACBP; ERP60; | |
| 52-kDa ribonucleoprotein autoantigen RO/SS-A | M84739 |
| transformation-sensitive protein IEF SSP 3521 | M86752 |
| alpha crystallin B subunit (alpha(B)-crystallin; CRYAB; CRYA2); | |
| Rosenthal fiber component | S45630 |
| heat shock protein beta 2 (HSPB2); DMPK-binding protein; MKBP | S67070 |
| alpha crystallin A chain (CRYAA; CRYA1) | U05569 |
| nicotinamide N-methyltransferase (NNMT) | U08021 |
| phenol-sulfating phenol sulfotransferase 1 (PPST1); | |
| thermostable phenol sulfotransferase (TS-PST); | |
| HAST1/HAST2; ST1A3; STP1 + PPST2; ST1A2; | |
| STP2 + monoamine-sulfating phenol sulfotransferase | U09031 + U28170 + |
| | L19956 |
| NADP + dihydropyrimidine dehydrogenase precursor (DPD); | |
| dihydrouracil dehydrogenase; dihydrothymine dehydrogenase (DPYD) | U09178 |
| transcriptional regulator atrX; X-linked helicase II (XH2); | |
| X-linked nuclear protein (XNP); RAD54L | U09820 |
| 26S proteasome regulatory subunit S2 (PSMD2); | |
| tumor necrosis factor type 1 receptor-associated protein (TRAP2); 55.11 protein | U12596 |
| damage-specific DNA-binding protein p127 subunit (DDBA p127); DDB1 | U18299 |
| T-complex protein 1 delta subunit (TCP1-delta); CCT-delta (CCTD; CCT4); | |
| stimulator of tar RNA binding (SRB) | U38846 |
| 7,8-dihydro-8-oxoguanine triphosphatase (8-oxo-dGTPase); mutT homolog 1 (MTH1) | D16581 |
| 150-kDa oxygen-regulated protein ORP150 | U65785 |
| 48-kDa FKBP-associated protein (FAP48) | U73704 |
| T-complex protein 1 eta subunit (TCP1-eta); CCT-eta (CCTH; CCT7); | |
| HIV-1 NEF interacting protein | U83843 |
| catalase (CAT) | X04076 |
| porphobilinogen deaminase (PBGD); hydroxymethylbilane synthase (HMBS); | |
| pre-uroporphyrinogen synthase | X04808 |
| Mn+ superoxide dismutase 2 precursor (SOD2) | X07834; X59445 |
| 94-kDa glucose-regulated protein (GRP94); endoplasmin precursor; GP96 homolog; | |
| tumor rejection antigen 1 (TRA1) | X15187; M33716 |
| uracil-DNA glycosylase 2 (UNG2) | X52486 |
| T-complex protein 1 alpha subunit (TCP1-alpha); CCT-alpha (CCTA; CCT1) | X52882 |
| 40S ribosomal protein S3 (RPS3) | X55715 |
| 47-kDa heat shock protein precursor; collagen-binding protein 1 (CBP1); | |
| colligin 1 + collagen binding protein 2 (CBP2) | X61598 + D83174 |
| T-complex protein 1 gamma subunit (TCP1-gamma); | |
| CCT-gamma (CCTG; CCT3); TRIC5 | X74801; U17104 |
| transcription factor IIH (TFIIH); 52-kDa basic transcription factor 2 subunit (BTF2p52) | Y07595 |
| x-ray repair cross-complementing protein 2 (XRCC2) | Y08837 |
| 8-oxyguanine DNA glycosylase 1 (OGG1); mutM homolog (MMH) | Y11838 |
| 34-kDa basic transcription factor 2 subunit (BTF2p34) | Z30093 |
| N-oxide forming dimethylaniline monooxygenase 5; | |
| hepatic flavin-containing monooxygenase 5 (FMO5); dimethylaniline oxidase 5 | L37080 |
| ubiquitin-like protein NEDD8 | D23662 |
| multidrug resistance protein 3 (MDR3); P-glycoprotein 3 (PGY3) | M23234 |
| ubiqitin-conjugating enzyme E2-17-kDa (UBE2B); ubiquitin-protein ligase; | |
| ubiquitin carrier protein; HR6B | M74525 |
| p59 protein; HSP-binding immunophilin (HBI); | |
| possible peptidyl-prolyl cis-trans isomerase (PPIase); | |
| rotamase; 52-kDa FK506-binding protein (FKBP52); | |
| FKBP59; HSP56; FKBP4 | M88279 |
| heat shock protein 40 homolog (HSP40 homolog); DNAJW | U40992 |
| 51-kDa FK506-binding protein (FKBP51); | |
| peptidyl-prolyl cis-trans isomerase (PPIase); | |
| rotamase; 54-kDa progesterone receptor-associated immunophilin; | |
| FKBP54; FF1 antigen; HSP90-binding immunophilin | U42031 |
| hematopoietic progenitor kinase (HPK1) | U66464 |
| SPS1/Ste20 homolog KHS1 | U77129 |
| liver glyceraldehyde 3-phosphate dehydrogenase (GAPDH; G3PDH) | X01677 |
| brain-specific tubulin alpha 1 subunit (TUBA1) | K00558 |
| HLA class I histocompatibility antigen C-4 alpha subunit (HLAC) | M11886 |
| cytoplasmic beta-actin (ACTB) | X00351 |
| 23-kDa highly basic protein; 60S ribosomal protein L13A (RPL13A) | X56932 |
| 40S ribosomal protein S9 | U14971 |
| ubiquitin | M26880 |
| phospholipase A2 | M86400 |
| hypoxanthine-guanine phosphoribosyltransferase (HPRT) | V00530 |

*sic; PNA oligomer?-Trans.
Note.

It is particularly preferred if the methylation state of essentially all of the genes listed in Table 1 is investigated.

A set of genes is preferably investigated for methylation in which up to 25% of the genes listed in Table 1 are not contained.

It is further preferred that at least 95% of the genes listed in Table 1 are investigated for their methylation state, together with a limited number of additional unlisted genes.

Up to 25% of the genes listed in Table 1 are replaced according to the invention by a complete set of other unlisted genes.

Preferably, the chemically pretreated DNA sequence of the genes to be detected coincides at least to 95% with the correspondingly pretreated DNA sequence of the genes from Table 1.

Sequences, which coincide 100% in a segment of the exon that is at least 25 base pairs long are designated homologous in this invention. This is true also for sequences whose homology is recognized only by taking into consideration possible frameshifts.

Several of the genes under investigation and their importance for toxicological processes are introduced in the following examples.

The genomic sequences of the genes under investigation can be derived by comparison of the respective cDNA sequences with publicly accessible databases, in which genomic sequences are deposited.

### EXAMPLE 1

### Culturing HT-29 P208 Cells, Harvesting Cells and Preparation of Chromosomal DNA

HT-29 P208 cells (5×10⁴ cells/ml) were seeded in culture dishes (33×5 cm) and grown for 5 days in DMEM/Ham's F-12 medium, which was supplemented with 10% fetal calf serum at 37° C. and 5% CO₂, up to 95% confluence (Campbell-Thompson, M. and Bhardwaj, B., Cancer Research 2001, 61, 632-640). The cells were then incubated for 24 h in medium not containing calf serum. After repeated change of medium, the cells were incubated in 3 parallel cultures for 6 h and 24 h, either with medium, medium supplemented with 10 ng/ml TGF-b1, medium supplemented with 10 ng/ml IL-1b, medium supplemented with trichostatin (50 nM) or medium supplemented with milrinone (50 μM). The cells freed of medium were treated with trypsin, centrifuged, and resuspended in 200 μl of PBS buffer (Fritsch and Maniatis eds., Molecular Cloning: A Laboratory Manual, 1989), and stored at −20° C. The chromosomal DNA was purified with a QIAamp Kit according to the manufacturer's instructions (Qiagen, Hilden).

### EXAMPLE 2

### Production of Bisulfite-Treated DNA and Conducting the PCR Reactions

The DNA samples (20 ng) were digested with the restriction enzyme Mss1. The digested DNA was chemically converted with hydrogen sulfite (bisulfite, disulfite) and a radical trap at elevated temperature (DE 10050942). In this way, after alkaline hydrolysis, all unmethylated cytosines are converted to uracil, which correspond in their base-pairing behavior to thymidine. In contrast, 5-methylcytosine is not modified under these conditions.

The chemically pretreated DNA was then amplified with the use of a heat-stable DNA polymerase in a polymerase chain reaction. The multiplex PCR reactions were conducted with a thermocycler (Eppendorf GmbH) with the use of 10 ng of bisulfite-treated DNA, each time with 6 μmol of primer oligonucleotides (mixture of up to 32 individual primer oligonucleotides; see Table 3), 800 μM dNTPs and 4.5 mM magnesium chloride. The cycler program was as follows: step 1, 14 min at 96° C.; step 2, 60 sec 96° C.; step 3, 45 sec 55° C.; step 4, 75 sec 72° C.; step 5, 10 min at 72° C.; steps 2 to 4 were repeated 39 times.

The DNA fragments of 64 different genes listed in Table 3 were amplified with 6 sets of multiplex PCR (mPCR) and with bisulfite-treated DNA as the template, as described above. The mPCR reactions (I, J, K, L, M, N) of the genomic, bisulfite-treated DNA were conducted with the combination of primer oligonucleotides as listed in Table 3. It is also possible, however, to use other primer oligonucleotides in the same way for the amplification of the genomic, bisulfite-treated DNA. The primer pairs listed in Table 1, however, are particularly preferred.

### EXAMPLE 3

### Determination of the Methylation State of Selected Genes

The amplificates produced in Example 2 were hybridized with 512 oligonucleotides, which were bound to a solid phase (Model, F. and Adorjian, P., Bioinformatics 2001, 17 Suppl. 1, 157-164). The solid phase loaded with oligonucleotides is named an oligonucleotide array below. The detectability of the hybridization product is based on primer oligonucleotides fluorescently labeled with Cy5, which were used for the amplification. A hybridization reaction of the amplified DNA with the oligonucleotide, for example, GTTTTTTTCGTTTTAGAG (Sequence ID 6), occurs only when a methylated cytosine is present at the said site of the bisulfite-treated DNA. Thus, the methylation state of the specific cytosine can be determined by means of the hybridization product. In order to verify the methylation state of said position, an oligonucleotide is found on the oligonucleotide array, which permits it to detect the unmethylated cytosine. This oligonucleotide is identical to the oligonucleotide which was previously used for the analysis of the methylated status of the sample, with the exception that the oligonucleotide bears a thymine base instead of a cytosine base at the position to be analyzed, e.g., GTTTTTTTTGTTTTAGAG (Sequence ID 7). Thus, a hybridization reaction occurs only if an unmethylated cytosine is present at the position to be analyzed.

The fluorescent signals are detected by scanning the oligonucleotide arrays with the fluorescence scanner Genpix 4000A (Axon Instruments, USA). The quantitative determination of the fluorescent signals is made with the analytical software Genepix 3.0 (Axon Instruments, USA).

In order to be able to assign methylation patterns to a specific treatment of the cells, the DNA methylation patterns of HT29-P208 cells grown with IL-1b (interleukin) or cells treated with TGF-b1 (transforming growth factor) were determined. The results obtained were stored in databases and CpG dinucleotides with different methylation states were identified. The methylation patterns were compared by cluster analyses and statististical methods (Model, F. and Adorjan, P., Bioinformatics 2001, 17 Suppl. 1, 157-164).

### EXAMPLE 4

### Change of the Methylation State in HT29 Cells Due to Exogenous Cytokines and Active Substances of Low Molecular Weight

In this Example, the PCR products (see Table 1) of a cell culture (see Example 1), were amplified with CyS-fluorescently labeled primers of bisulfite-treated DNA, mixed, and hybridized on glass slides on which a pair of immobilized oligonucleotides was placed at each position. Each of these detection oligonucleotides was designed to hybridize the bisulfite-converted sequences found at CpG sites, which had been present originally in either the unmethyled (TG) or methyled (CG) state. The hybridization conditions were selected so as to display the detection of differences of the TG and CG variants in the individual nucleotides. The ratios of the two signals were calculated on the basis of a comparison of the intensities of the fluorescing signals.

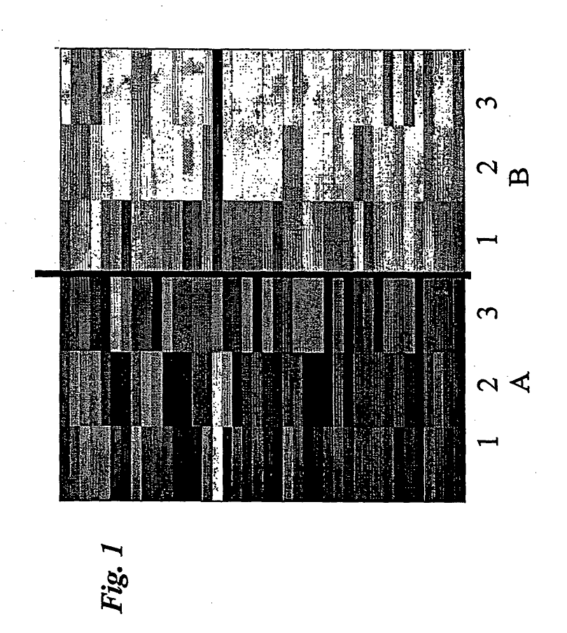

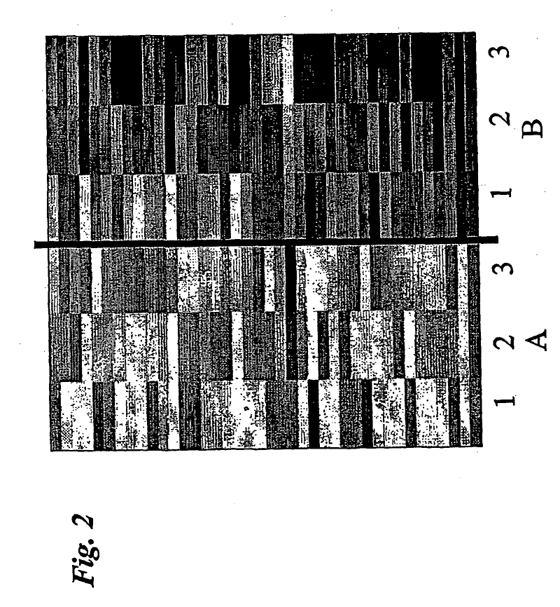

The information is subsequently defined in a weighted matrix (see FIGS. 1, 2) for the differences in CpG methylation between two classes of HT29-P208 cells: treated and untreated. The p-value for weighted methylation (p<0.05, F. Model, P. Adorjan, A. Olek, C. Piepenbrock, Feature selection for DNA methylation based cancer classification. Bioinformatics. 2001 June; 17 Suppl 1:S157-64) shows a clear difference between the two groups, which can be recognized from the variable gray shading. A higher degree of methylation correlates with a darker gray value.

Comparative investigations of the methylation state of 64 gene fragments (see Table 1) of HT29-P208 cells showed that both IL-1b as well as also TGF-b1 lead to a change of the methylation state of specific genes. In contrast to the controls (HT29-P208 in medium), a smaller degree of methylation could be found by supplementing the medium with TGF-b1, while a greater degree of methylation of various CpG-positions could be found by supplementing the medium with IL-1b. The changes in the methylation state were visible after a treatment time of 24 h (see FIGS. 1 and 2). After 6 h of treatment time no significant differences in methylation could be detected (data not shown). With the exception of CpG positions of the TGF-a gene, TGF-b1 and IL-1b change the methylation state of different genes.

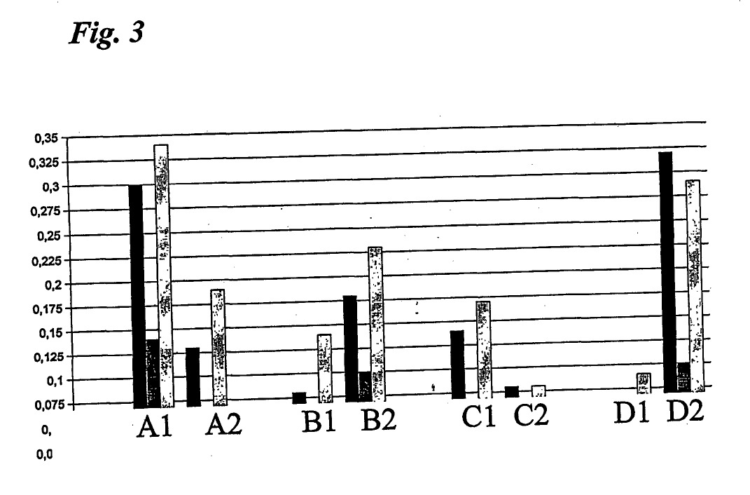

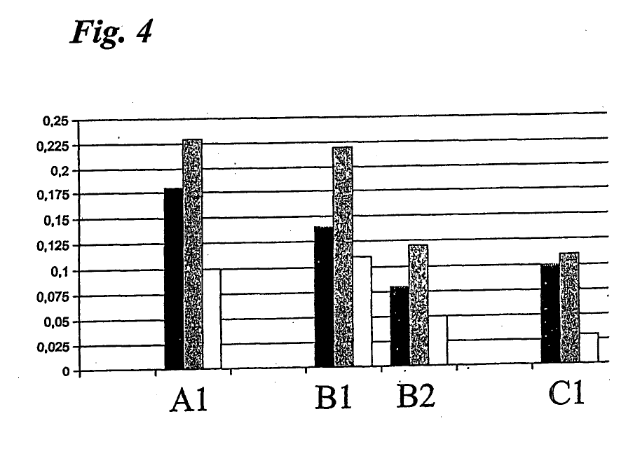

FIG. 3 shows quantitatively the methylation state of selected CpGs for the genes TGF-a, EGFR, ANT1 and E-cadherin. The amplification of these genes is performed under the conditions described in Example 2. Table 2 summarizes the primer sequences used, their Seq ID, the length of the PCR fragment, as well as the oligonucleotides used for the methylation analysis with their Seq ID. The methylation state was determined by the calculation of the quotient of the fluorescent signal of the CG oligos over the sum of the fluorescent signals of both the TG and CG oligos. Thus, the methylation state can vary between 0 and 1, whereby 0 indicates the minimum and 1 indicates the maximum methylation state. For the CpG positions investigated here, a significant reduction of the methylation state was determined in HT29-P208 cells due to the treatment with TGF-b1 (see FIG. 3). In contrast, treatment with IL-1b led to no change or to an increase in the methylation state (see FIG. 3, A1 and 2, B1 and 2, D1). In another experiment, the effect of milrinone and trichostatin on the methylation state of selected CpG positions of the genes EGFR, ANT1 and CDC25A was investigated. The treatment of HT29-P208 cells with milrinone led to a reduction in the methylation state, but an increase occurred with trichostatin (see FIG. 4).

### EXAMPLE 5

### Methylation Analysis of the Cyp1a1 Gene

Genes which code for enzymes that catalyze the biotransformation of toxicological substances are particularly preferred in the analysis of modified methylation patterns, which in turn reflect changes in gene expression. A central function has been imputed in this way, among others, to the genes of the cytochrome P450 family. Among other things, it could be shown in animal experiments that one of the genes of this class, Cyp1a1, was induced after mice were exposed to beta-naphthoflavone (Arch Biochem Biophys Apr. 1, 2000;376(1):66-73). First, the genomic DNA sequence that encodes the Cyp1a1 gene must be identified for the methylation analysis. For this purpose, for example, the cDNA sequence (Genbank Acc. No. 000499) will be compared with a genomic database (e.g. Genbank htgs) whereby usually the BLAST comparison algorithm, which is available via the Internet (www.ncbi.nlm.nih.gov), is used. Therefore, in the present case, the segment of genomic DNA, which codes for Cyp1a1, can be identified (Genbank Acc. AC020705). Preferably, genomic segments in the region of the promoter as well as of the first exon or intron are investigated for differences in methylation, since relevant CpG dinucleotides, whose methylation influences gene expression, can preferably be found in these segments. In the example of the Cyp1a1 gene, exon 1 (underscored) was localized in the following genomic sequence segment:

>genomic region, which contains exon 1 of the Cyp1a1 gene (exon 1 in bold letters)

>genomic region, which contains exon 1 of the
Cyp1a1 gene (exon 1 in bold letters)
catgccaaatggcactggggcttcgtgtcgtgccacagcgtggaccgaaa
atgcggacacatgcaggctgcctctcctcgcaggcagaagccacacgcag
acctagaccctttgcaccgcatccccttattcaatcgcgcacccgccacc
cttcgacagttcctctccctccaccccaaccccacgccgcgcgcgaggct
ggccctttaagagccccgccccgactccctcccccctcgcgtgactgcga
gcccccgcgccgggccggggaatgggtcggctgggtggctgcgcgggcct
ccggtccttctcacgcaacgcctgggcaccgcgcctccgggccaggtggg
gcggggacgggccgcctgacctctgccccctagagggatgtcgccggcgc
acgcaagctagccgggggtagggtgggggctccgcgccaggtgccccctc
cgtggtccctgggcccgagtctttccgtggccccccgccgccggatttct
gtgctctgccaatcaaagcactagccaccccgggagccaagagggaccct
caagggccggtgggtcctggctggagggaccgcgcgttgcaatcagcact
aaggcgatcctagaggctgcgaggagccgctagtgagcgctcagcgagcc
tgccccttcgccatccattccgatccttcaatcaagaggcgcgaacctca
gctagtcgcccgggctctgggggacaggtccagccccgcggcgcctctgg
ccttccggcccccgtgacctcagggctggggtcgcagcgcttctcacgcg
agccgggactcagtaaccccgggaaggaggtcaccacggggcagccccgc
ccccgcctgccgagtcctggtaggctgtagcgctggggaggcatctgcac
gcccagcgttccagtgggtgcaaaaatgacgaagaggagtccccgcgccc
caggatggagcttcccgtaccctctcttcgggctgtcctgggacttctcc
ctcaagccccctcctcggctgggttctgcactgcccttgggacgccttgg
aattgggacttccaggtgttcccagccctcacccctctatgtacaggcac
cgagatgtgtcccatagtgggttcttgcccacccgaccccccacccccgc
cgccctccgccacctttctctccaatcccagagagaccagcccggttcag
gctgcttctccctccatctcagctcgctccagggaaggaggcgtggccac
acgtacaagcccgcctataaaggtgcagtacttcaccctcaccctgaagg
tgacagttcttggatgttccctgatccttgtgatcccaggctccaagagt
ccacccttcccagctcagctcagtacctcaggtgagttgctgggggactt
ctggcttgccctttctctcccaataaaaggaacattttggtgcctccagg
acttcttaggtagctacctgtctagcacctccaaaaagggaggctcagag
tgtttttagtgaccaggcagtctagccccctagtggggaaactgaggcca
ggggaagaggaggacttgcccatggtcccacagctggtaccagacctcta
gatacagatggcatctcattcaggacttcaggacccaggctccagctcca
tcccctagtgagtgtctccctgcctaccctggggcttccccatcaaggcc
acctggcaggctggaatatgtgcagcccctccctcaggctttctgatgac
aggggcttctccttgggtggacagggtggatggagggggtgggctgggtt
cttaccagctgtaccctgccctagcctaagaagctacccctggcagattt
taccctcctaagggtggcttgtcagtgctgagatgtcctagacagctggg
acaatagaggcagatctgtgcaggagtcccaggcctttcctatctcattg
accatcttctttgtcctttgctgggagacaatcagggtgacagattgcca
actgcagggagctggaaataccagtccctaaaaactcaccagtcacatct
cccttggcctcctaccatcttacaaaggctccagctccatcccctagtga
gtgtctccctgcctaccctggggcttccccataaagccacctggcaggct
ggaatatgtgcagcccctccctcaggctttctgatgacagcccctccagg
aagcctcccctccactataatacttgtggtaggaaccatccatctccctg
tcttgtgaggttctcctgtggggagcctaactggtaagactgtcattccc
cacagcagatctgggttttctcttccctctggatccagctgggtactctg
aactgagagatcttgtcttaccctctctcagatgttgaaattggacccca
gaaaagtaaaatgtgcagtccaagatcactttgactagaatgttggtcta
ctgacctctagtccagggtaacaggcagagatgcctgatatgttggagag
agtggtttatgaatttaaacaccctttttaggtcaagcttacagagaaag
tattgcctcagtttcctttcagtttagatccattcctgatttccctgatt
ccagtctggggttttcttacagcctagtgggaaccttccatttattctct
gctctctggtaacctgcaaaagggggaggtccaaactgttcattcattga
gaa

Sequence segment after bisulfite treatment and PCR amplification (the primer oligonucleotides used and the analyzed CpG are given in bold letters)

>cyp1a1 sense-1 bisulfite
tatgttaaatggtattggggtttCGtgtCGtgttatagCGtggatCGaaa
atgCGgatatatgtaggttgttttttttCGtaggtagaagttataCGtag
atttagattttttgtatCGtatttttttatttaatCGCGtattCGttatt
tttCGatagtttttttttttttattttaattttaCGtCGCGCGCGaggtt
ggttttttaagagtttCGtttCGatttttttttttttCGCGtgattgCGa
gttttCGCGtCGggtCGgggaatgggtCGgttgggtggttgCGCGggttt
tCGgttttttttaCGtaaCGtttgggtatCGCGttttCGggttaggtggg
gCGgggaCGggtCGtttgatttttgttttTtagaGggatgtCGtCGgCGt
aCGtaagttagtCGggggtagggtgggggtttCGCGttaggtgttttttt
CGtggtttttgggttCGagttttttCGtggtttttCGtCGtCGgattttt
gtgttttgttaattaaagtattagttatttCGggagttaagagggatttt
taagggtCGgtgggttttggttggagggatCGCGCGttgtaattagtatt
aaggCGattttagaggttgCGaggagtCGttagtgagCGtttagCGagtt
tgttttttCGttatttatttCGattttttaattaagaggCGCGaatttta
gttagtCGttCGggttttgggggataggtttagtttCGCGgCGtttttgg
tttttCGgttttCGtgattttagggttggggtCGtagCGttttttaCGCG
agtCGggatttagtaatttCGggaaggaggttattACGGGgtagtttCGt
tttCGtttgtCGagttttggtaggttgtagCGttggggaggtatttgtaC
GtttagCGttttagtgggtgtaaaaatgaCGaagaggagttttCGCGttt
taggatggagtttttCGtatttttttttCGggttgttttgggattttttt
tttaagtttttttttCGgttgggttttgtattgtttttgggaCGttttgg
aattgggatttttaggtgtttttagtttttatttttttatgtataggtat
CGagatgtgttttatagtgggtttttgtttattCGattttttattttCGt
CGtttttCGttatttttttttttaatttagagagattagttCGgtttagg
ttgtttttttttttattttagttCGttttagggaaggaggCGtggttata
CGtataagttCGtttataaaggtgtagtattttatttttattttgaaggt
gatagtttttggatg

In order to be able to amplify a part of the genomic segment shown after the bisulfite treatment, for example, the two primers with the sequences TATGTTAAATGGTATTGG and CATCCAAAAACTAT are used, with which defined fragments with a length of 1316 base pairs can be amplified. These amplificates serve as probes, which will be hybridized to oligonucleotides that have previously been bound to a solid phase, for example, CTACCCCGTAATA, whereby the cytosine to be detected is found at position 837 of the amplificate. The detection of the hybridization product is based on primer oligonucleotides fluorescently labeled with Cy3 or Cy5, which were used for the amplification. A hybridization reaction of the amplified DNA with the oligonucleotide occurs only if a methylated cytosine was present at this site in the bisulfite-treated DNA. Thus, the methylation state of the respective cytosine to be investigated decides the hybridization product.

### EXAMPLE 6

### Classification of a Chemical Substance in a Toxicity Class by Determination of the Methylation Pattern

In order to assign a chemical substance to a specific class on the basis of the methylation pattern, first an investigation must be made of the DNA methylation patterns in a group of exposed and a group of unexposed organisms, for example, experimental animals. The results are stored in a database and CpG dinucleotides are identified which are differently methylated in the two groups. Then the methylation pattern for the substance to be evaluated is compared with already known methylation patterns of other chemical substances. Indications of the properties of the substance to be investigated can be obtained from the toxicological properties of those chemical substances with similar methylation pattern.

The subject of the present invention is also a kit comprising a bisulfite-containing reagent, a set of primer oligonucleotides comprising at least two oligonucleotides, each of whose sequence of a segment at least 18 base pairs long corresponds to the base sequence of the genes to be investigated, or is complementary to them, for the production of amplificates, oligonucleotides and/or PNA oligomers, a control nucleic acid, as well as instructions for conducting and evaluating the described method.

Legends to Figures:

FIG. 1

Weighted matrix of the methylation state (fluorescent signal CG oligo×(fluorescent signal CG oligo+fluorescent signal TG oligo)−1) of 40 CpGs in untreated HT29-P208 cells (A) and TGF-b1-treated HT29-P208 cells (B). The numbers 1-3 denote 3 independent experiments (cell treatments and methylation analysis). Each horizontal row represents one CpG, whose methylation state is different, with a significance of p<0.05, in the two groups analyzed. The darker gray tones correspond to a higher degree of methylation, while the lighter gray tones correspond to a smaller degree of methylation.

FIG. 2

Weighted matrix of the methylation state (fluorescent signal CG oligo×(fluorescent signal CG oligo+fluorescent signal TG oligo)−1) of 40 CpGs in untreated HT29-P208 cells (A) and IL-1b-treated HT29-P208 cells (B). The numbers 1-3 denote 3 independent experiments (cell treatments and methylation analysis). Each horizontal row represents one CpG, whose methylation state is different, with a significance of p<0.05, in the two groups analyzed. The darker gray tones correspond to a higher degree of methylation, while the lighter gray tones correspond to a smaller degree of methylation.

FIG. 3

Quantitative analysis of the methylation state (fluorescent signal CG oligo×(fluorescent signal CG oligo+fluorescent signal TG oligo)−1) of 8 CpGs in untreated HT29-P208 cells (black columns), TGF-b1-treated (gray columns) and IL-1b-treated (white columns) HT29-P208 cells. CpGs, which are represented by the indicated oligo SEQ IDs were investigated from the following genes: TGF-a (A1, oligo SEQ IDs 6, 7; A2, oligo SEQ IDs 8, 9), EGFR (B1, oligo SEQ IDs 20, 21; B2, oligo SEQ IDs 22, 23), ANT1 (C1, oligo SEQ IDs 32, 33; C2, oligo SEQ IDs 34, 35) and E-cadherin (D1, oligo SEQ IDs 13, 14; D2, oligo SEQ IDs 15, 16). The numerical values of the y-axis show the methylation state, calculated as the quotient of the fluorescent signal of the CG oligos over the sum of the fluorescent signals of both the TG and CG oligos.

FIG. 4

Quantitative analysis of the methylation state (fluorescent signal CG oligo×(fluorescent signal CG oligo+fluorescent signal TG oligo)−1) of 4 CpGs in untreated HT29-P208 cells (black columns), trichostatin-treated (gray columns) and milrinone-treated (white columns) HT29-P208 cells. CpGs, which are represented by the indicated oligo SEQ IDs, were investigated from the following genes: EGFR (A1, oligo SEQ IDs 22, 23), ANT1 (B1, oligo SEQ IDs 32, 33; B2, oligo SEQ IDs 34, 35) and CDC25A (C1, oligo SEQ IDs 27, 28). The numerical values of the y-axis show the methylation state, calculated as the quotient of the fluorescent signal of the CG oligos over the sum of the fluorescent signals of both the TG and CG oligos.

1. A method for toxicological diagnosis is hereby characterized in that the following method steps are conducted:
a) a sample that contains the DNA of an organism or a cell culture is taken from this organism or cell culture, which has previously been subjected to a specific substance to be tested for its toxicological effect;
b) the DNA contained in the sample is treated in such a way that methylated cytosine bases lead to a base sequence at a given position in said treated DNA that is different than unmethylated cytosine bases at a given position in said treated DNA;
c) the base sequence of a part of the modified DNA is determined and from this a methylation state is concluded that is characteristic for the sample or a characteristic methylation pattern is obtained;
d) the effect of a substance on the organism or the cell culture is concluded by comparison with data of the methylation states of other samples and/or the effect of this substance on the organism or the cell culture is compared with other substances from a toxicological point of view.

2. A method for the toxicological diagnosis according to claim 1, further characterized in that the methylation state or the methylation pattern of a set of genes is investigated, which comprises at least one of the genes listed in the following, or sequences which are identical or at least 85% homologous in the region of the exon to the genes listed below:

| | GenBank Accession |
|---|---|
| Gene Name | #(s) |
| serotransferrin precursor; siderophilin; beta-1-metal binding globulin | M12530 |
| lactotransferrin precursor; lactoferrin | X53961 |
| apolipoprotein E precursor (APOE) | M12529 |
| lipopolysaccharide-binding protein precursor (LBP) | M35533 |
| B-lymphocyte kinase; tyrosine-protein kinase BLK; p55-BLK | Z33998 |
| apolipoprotein A-I precursor (APOAI) | X00566 |
| apolipoprotein A-II precursor (APOAII) | X00955 |
| apolipoprotein C-III precursor (APOCIII) | X01388 |
| endothelin 1 (ET1) | Y00749 |
| macrophage colony stimulating factor 1 (CSF1; MCSF) | M37435 |
| familial intrahepatic cholestasis 1 protein (FIC1) | AF038007 |
| vascular endothelial growth factor D (VEGFD); C-FOS-induced growth factor (FIGF) | D89630 |
| complement component 4-binding protein alpha (C4B-binding protein; C4BPA); | M31452 |
| proline-rich protein (PRP) | |
| insulin-like growth factor II (IGF2); somatomedin A | M29645 |
| granulocyte-macrophage colony stimulating factor (GM-CSF); CSF2 | M11220 |
| epidermal growth factor precursor (EGF); beta-urogastrone | X04571 |
| hepatocyte growth factor activator (HGF activator) | D14012 |
| macrophage inflammatory protein 1 beta precursor (MIP1-beta); | J04130 |
| T-cell activation protein 2 (AT2); PAT 744; H400; SIS-gamma; | |
| lymphocyte activation gene 1 protein (LAG 1); HC21; | |
| small inducible cytokine A4 (SCYA4); G 26 T-lymphocyte secreted protein | |
| glial growth factor 2 precursor (GGFHPP2); neuregulin; heregulin-beta3 + neu | L12260; L12261 + |
| differentiation factor + heregulin-alpha | U02326 + M94165 |
| T-cell-specific rantes protein precursor; sis delta; small inducible cytokine A5 (SCYA5); | M21121 |
| rantes pro-inflammatory cytokine | |
| macrophage inflammatory protein 1 alpha precursor (MIP1-alpha); | M23452 |
| tonsillar lymphocyte LD78 alpha protein; G0S19-1 protein; PAT 464.2; SIS-beta; | |
| small inducible cytokine A3 (SCYA3) | |
| oncostatin M (OSM) | M27288 |
| insulin-like growth factor binding protein 1 (IGFBP1); placental protein 12 (PP12) | M31145 |
| vascular endothelial growth factor precursor (VEGF); | M32977; M27281 |
| vascular permeability factor (VPF) | |
| hepatocyte growth factor (HGF); scatter factor (SF); hepatopoeitin A | M60718 |
| thymosin beta-10 (TMSB10; THYB10); PTMB10 | M92381 |
| interferon gamma-induced protein precursor (gamma-IP10) | X02530 |
| macrophage inflammatory protein 2 alpha (MIP2-alpha); | X53799 |
| growth-regulated protein beta (GRO-beta) | |
| OX40 ligand (OX40L); GP34; tax-transcriptionally activated glycoprotein 1 (TXGP1) | X79929 |
| transforming growth factor-beta 3 (TGF-beta3) | J03241 |
| delta-like protein precursor (DLK) | U15979; Z12172 |
| insulin-like growth factor IA precursor (IGF1A); IGFBP1; | M27544 + M37484 |
| somatomedin C + insulin-like growth factor I (IGF1) | |
| CC chemokine eotaxin precursor; eosinophil chemotactic protein; | D49372; Z75669; Z75668 |
| small inducible cytokine A11 (SCYA11) | |
| sonic hedgehog (SHH) | L38518 |
| interleukin-1 receptor antagonist protein precursor (IL-1RA; IRAP) | M63099 |
| macrophage inhibitory cytokine 1 (MIC1) | AF019770 |
| erythropoietin | M11319 |
| eosinophil granule major basic protein precursor (MBP); | Y00809 |
| pregnancy-associated major basic protein; bone marrow proteoglycan 2 | |
| insulin-like growth factor-binding protein 3 precursor | |
| (IGF-binding protein 3; IGFBP3; IBP3) | M31159; M35878 |
| cellular retinoic acid-binding protein II (CRABP2) | M68867 |
| corticoliberin precursor; corticotropin-releasing factor (CRF); | V00571 |
| corticotropin releasing hormone (CRH) | |
| interferon gamma precursor (IFN-gamma; IFNG); immune interferon | X01992; M29383 |
| interleukin-2 precursor (IL-2); T-cell growth factor (TCGF) | A14844 |
| interleukin-1 alpha precursor (IL-1 alpha; IL1A); hematopoietin-1 | X02851 |
| interleukin-4 precursor (IL-4); B-cell stimulatory factor 1 (BSF-1); | M13982 |
| lymphocyte stimulatory factor 1 | |
| interleukin-6 precursor (IL-6); B-cell stimulatory factor 2 (BSF2); | X04602; M14584 |
| interferon beta-2 (IFNB2); hybridoma growth factor | |
| interleukin-5 precursor (IL-5); T-cell replacing factor (TRF); | X04688; J03478 |
| eosinophil differentiation factor; B-cell differentiation factor I | |
| interleukin-12 beta subunit precursor (IL-12B); | M65290 |
| cytotoxic lymphocyte maturation factor 40-kDa subunit (CLMF p40); | |
| NK cell stimulatory factor subunit 2 (NKSF2) | |
| interleukin-12 alpha subunit precursor (IL-12A); | M65291 |
| cytotoxic lymphocyte maturation factor 35-kDa subunit (CLMF p35); | |
| NK cell stimulatory factor subunit 1 (NKSF1) | |
| pancreatitis-associated protein 1 precursor | D13510 |
| alpha-1-acid glycoprotein 1 precursor (AGP1); orosomucoid 1 (OMD1) | X02544 |
| C-reactive protein precursor | X56692 |
| corticosteroid-binding globulin | J02943 |
| prostaglandin-endoperoxide synthase 1 precursor; prostaglandin G/H synthase1 | |
| (PGH synthase 1; PTGS1; PHS1); cyclooxygenase 1 (COX1) | M59979 |
| amphiphysin (AMPH) | U07616 |
| 5-hydroxytryptamine 1D receptor (5-HT-1D; HTR1D); serotonin receptor | M89955 |
| neuromedin B precursor | M21551 |
| major prion protein precursor (PRP); PRP27-30; PRP33-35C; ASCR | M13667 |
| dopamine beta-hydroxylase (DBH); dopamine-beta-monooxygenase precursor | X13255 |
| Alzheimer's disease amyloid A4 protein precursor; protease nexin-II (PN-II); APPI | Y00264 |
| membrane-bound & soluble catechol-O-methyltransferase (COMT) | M65212 |
| flavin-containing amine oxidase A; monoamine oxidase (MAO-A) | M68840 |
| erythropoietin receptor (EPOR) | M60459 |
| cation-independent mannose-6-phosphate receptor precursor | |
| (CI man-6-P receptor; CI-MPR); | Y00285; J03528 |
| insulin-like growth factor II receptor (IGFR II) | |
| activin receptor type II precursor (ACTRIIA; ACVR2) | D31770 |
| RETINOID X RECEPTOR GAMMA (RXR-GAMMA) | U38480 |
| transcriptional enhancer factor (TEF1); protein GT-IIC; transcription factor 13 (TCF13) | M63896 |
| glucocorticoid receptor (GRL) | M10901 |
| orphan nuclear hormone receptor BD73 | L31785 |
| low-density lipoprotein receptor (LDL receptor; LDLR) | M28219 |
| sulfonylurea receptor 2A (SUR2A) | AF061323 |
| sulfonylurea receptor (SUR); | L78207 |
| ATP-binding cassette subfamily C (CFTR/MRP) member 8 (ABCC8) | |
| farnesol receptor HRR-1 | U68233 |
| tyrosine protein kinase receptor UFO | X66029 |
| colorectal cancer suppressor protein precursor (DCC) | X76132 |
| vascular cell adhesion protein 1 | X53051 |
| alpha1 catenin (CTNNA1); cadherin-associated protein; | D13866; D14705; L23805; |
| alpha E-catenin | L22080 |
| integrin alpha 9 (ITGA9); integrin alpha-RLC | D25303; L24158 |
| intercellular adhesion molecule 1 precursor (ICAM1); major group rhinovirus receptor; | J03132 |
| CD54 antigen | |
| ras-related protein RAB5A | M28215 |
| E-selectin precursor (SELE); endothelial leukocyte adhesion molecule 1 (ELAM1); | M30640 |
| leukocyte-endothelial cell adhesion molecule 2 (LECAM2); CD62E antigen | |
| NADH-ubiquinone dehydrogenase 1 beta subcomplex 7 18-kDa subunit (NDUFB7); | M33374 |
| complex I-B18 (CI-B18); cell adhesion protein SQM1 | |
| neural-cadherin precursor (N-cadherin; NCAD); | M34064; X57548; X54315; |
| cadherin 2 (CDH2) | S42303 |
| cell surface adhesion glycoproteins LFA-1/CR3/p150,95 beta-subunit precursor; | M15395 |
| LYAM1; integrin beta 2 (ITGB2); CD18 antigen; complement receptor C3 beta subunit | |
| fibronectin receptor alpha subunit (FNRA); integrin alpha 5 (ITGA5); VLA5; | X06256 |
| CD49E antigen | |
| fibronectin receptor beta subunit (FNRB); integrin beta 1 (ITGB1); | X07979 |
| very late antigen 4 beta subunit (VLA4); CD29 antigen | |
| integrin alpha L (ITGAL); leukocyte adhesion glycoprotein alpha subunit precursor; | Y00796 |
| leukocyte function-associated molecule 1 alpha chain (LFA1); CD11A antigen | |
| cadherin 6 precursor (CDH6); kidney cadherin (K-cadherin) | D31784 |
| cadherin 11 precursor (CDH11); osteoblast-cadherin (OB-cadherin); OSF4 | L34056 |
| cadherin 12 (CDH12); brain cadherin precursor (Br-cadherin); | L34057; L33477 |
| neural cadherin 2 (N-cadherin 2) | |
| cadherin 13 (CDH13); truncated cadherin precursor (T-cadherin); | L34058; U59289; |
| heart cadherin (H-cadherin) | |
| U59288 | |
| cadherin 3 (CDH3); placental cadherin precursor (P-cadherin; CDHP) | X63629 |
| GAP JUNCTION ALPHA-5 PROTEIN (CONNEXIN 40) (CX40) | L34954 |
| INVOLUCRIN | M13903 |
| fibrinogen G gamma polypeptide | X51473; X02415 K02569 |
| plasma-cell membrane glycoprotein PC-1; alkaline phosphodiesterase I; | M57736 |
| nucleotide pyrophosphatase (NPPase) | |
| annexin V; lipocortin V; endonexin II; calphobindin I (CBP-I); | X12454 |
| placental anticoagulant protein I (PAP-I); PP4; thromboplastin inhibitor; | |
| vascular anticoagulant-alpha (VAC-alpha; anchorin CII | |
| laminin alpha 1 subunit precursor (LAMA1); laminin A chain | X58531 |
| intestinal fatty acid-binding protein 2 (FABP2; IFABP) + liver | M10050 + M10617 |
| fatty acid-binding protein 1 (FABP1; LFABP) | |
| sodium-independent organic anion transporter; | U21943 |
| organic anion transporting polypeptide (OATP); SLC21A3 | |
| polyspecific organic cation transporter N1 (OCTN1) | AB007448 |
| TNF-alpha-stimulated ABC protein (TSAP) | AF027302 |
| organic cation transporter-like protein 2 (ORCTL2) | AF037064 |
| organic cation transporter N2 (OCTN2) | AF057164 |
| MRP/organic anion transporter (MOAT-B) | AF071202 |
| adrenoleukodystrophy-related protein (ALDR) | AJ000327 |
| skeletal muscle adenine nucleotide translocator 1 (ANT1); | J02966 |
| heart/skeletal muscle ADP/ATP carrier protein isoform T1; | |
| ADP/ATP translocase 1 | |
| down-regulated in adenoma protein (DRA) | L02785 |
| mitochondrial uncoupling protein 3 (UCP3) | AF011449 |
| mitochondrial carnitine palmitoyltransferase II precursor (CPTase; CPT2) | M58581 |
| mitochondrial brown fat uncoupling protein 1 (UCP1) | U28480 |
| prostaglandin transporter (PGT); solute carrier family 21 member 2 (SLC21A2) | U70867 |
| mitochondrial uncoupling protein 2 (UCP2); UCPH | U82819 |
| bile salt export pump (BSEP) | AF091582 |
| anthracycline resistance-associated protein (ARA) | X95715 |
| kidney organic cation transporter | X98333 |
| multidrug resistance-associated protein 3 (MRP3); MLP2; ABCC3 | Y17151 |
| antigen peptide transporter 2 (APT2); peptide supply factor 2 (PSF2); | X66401; L09191; L10287 |
| peptide transporter involved in antigen processing 2 (TAP2); | |
| ATP-binding cassette subfamily B (MBR/TAP) member 3 (ABCC3); | |
| HLA class II histocompatibility antigen DO beta chain precursor | |
| putative renal organic anion transporter 1 (hROAT1) | AF057039 |
| chloride conductance regulatory protein ICLN; nucleotide-sensitive chloride channel 1A; | X91788 |
| chloride ion current inducer protein (CLCI); reticulocyte PICLN | |
| neutral amino acid transporter A (SATT); | L14595 |
| alanine/serine/cysteine/threonine transporter (ASCT1) | |
| monocarboxylate transporter 1 (MCT1) | L31801 |
| ileal sodium-dependent bile acid transporter (ISBT); | U10417 |
| ileal sodium/taurocholate cotransporting polypeptide (NTCP2); SLC10A2 | |
| sodium-dependent bile acid cotransporter; | L21893 |
| hepatic sodium/taurocholate cotransporting polypeptide (NTCP); SLC10A1 | |
| sodium-& chloride-dependent glycine transporter 1 (GLYT-1) | S70609 |
| cystic fibrosis transmembrane conductance regulator (CFTR); | M28668 |
| cAMP-dependent chloride channel | |
| canalicular multispecific organic anion transporter; | U63970 |
| multidrug resistance-associated protein 2 (MRP2); | |
| canalicular multidrug resistance protein | |
| organic cation transporter 1 | U77086 |
| GAP JUNCTION BETA-1 PROTEIN (CONNEXIN 32) | X04325 |
| (CX32) (LIVER GAP JUNCTION PROTEIN) | |
| cadherin1 (CDH1); epithelial cadherin precursor (E-cadherin; CDHE); | Z13009 |
| uvomorulin (UVO); CAM 120/80 | |
| smoothened; GX | U84401 |
| ephrin type-A receptor 2 precursor; epithelial cell kinase (ECK); | M59371 M36395 |
| tyrosine-protein kinase receptor ECK | |
| NADPH-cytochrome p450 reductase | S90469 |
| NCK melanoma cytoplasmic src homolog (HSNCK) | X17576 |
| JV18-1. HMAD-2 OR MADR2 OR SMAD2 | U68018 |
| dual-specificity mitogen-activated protein kinase kinase 1 (MAP kinase kinase 1; | L05624 |
| MAPKK 1; MKK1); extracellular signal-regulated kinase 1; | |
| ERK activator kinase 1 | |
| c-jun N-terminal kinase 1 (JNK1); JNK46 | L26318 |
| mitogen-activated protein kinase p38 (MAP kinase p38); | |
| cytokine suppressive anti-inflammatory drug-binding protein | |
| (CSAID binding protein; CSBP); MAX-interacting protein 2 (MXI2) | L35253; L35263 |
| protein kinase C beta I (PKC-beta-1) | M27545; X06318 |
| mitogen-activated protein kinase 9 (MAP kinase 9; MAPK9; PRKM9); | L31951 |
| c-jun N-terminal kinase 2 (JNK2); JNK55 | |
| C-jun N-terminal kinase 3 alpha2 (JNK3A2); PRKM10 + MAP kinase p493F12 | U34819 + U07620 |
| dual-specificity mitogen-activated protein kinase kinase 6 | |
| (MAP kinase kinase 6; MAPKK 6; MKK6); MAPK/ERK kinase 6; SAPKK3 | U39657 |
| p21-activated kinase gamma (PAK-gamma; PAK2); PAK65; S6/H4 kinase | U24153 |
| mitogen-activated protein kinase P38 beta (MAP kinase P38 beta); | U53442 |
| stress-activated protein kinase 2 (SAPK2) | |
| MAPK/ERK kinase kinase 3 (MEK kinase 3; MEKK3) | U78876 |
| dual specificity mitogen-activated protein kinase kinase 2 | |
| (MAP kinase kinase 2; MAPKK 2); ERK activator kinase 2; | L11285 |
| MAPK/ERK kinase 2 (MEK2) | |
| dual specificity mitogen-activated protein kinase kinase 5 (MAP kinase kinase 5; | U25265 |
| MAPKK 5) | |
| ribosomal protein S6 kinase II alpha 1 (S6KII-alpha 1); ribosomal S6 kinase 1 (RSK1) | L07597 |
| B-lymphocyte germinal center kinase (GC kinase) | U07349 |
| YSK1; Ste20 & SPS1-related kinase | D63780 |
| protein phosphatase 2B regulatory subunit; calcineurin B subunit isoform 1 | M30773 |
| protein-tyrosine phosphatase MEG2 (PTPASE-MEG2) | M83738 |
| protein-tyrosine phosphatase alpha precursor (R-PTP-alpha; PTPRA; PTPA) | M34668 |
| ras associated with diabetes (RAD1) | L24564 |
| CDC42 homolog; G25K GTP-binding protein | M35543 + M57298 |
| (brain isoform + placental isoform) | |
| calmegin | D86322 |
| calbindin; avian-type vitamin D-dependent calcium binding protein (CABP); | D-28K X06661 |
| stratifin (SFN); 14-3-3 protein sigma; epithelial cell marker protein 1; HME1 | AF029082 |
| FKBP-rapamycin associated protein (FRAP); rapamycin target protein | L34075 |
| zinc finger protein 37 (ZFP37); KRAB domain zinc finger protein | AF022158 |
| CCAAT/enhancer-binding protein epsilon (C/EBP epsilon; CEBPE) | U48866; U48865 |
| transcription initiation factor IID; TATA-box factor; | M34960 |
| TATA sequence-binding protein (TBP) | |
| 60S ribosomal protein L6 (RPL6); TAX-responsive enhancer element binding protein 107 (TAXREB107); | X69391 |
| neoplasm-related protein C140 | |
| DNA-binding protein HIP116; ATPase; SNF2/SWI2-related protein | L34673 |
| basic transcription factor 2 44-kDa subunit (BTF2p44) | Z30094 |
| octamer-binding transcription factor 2 (oct-2; OTF2); lymphoid-restricted | M36542 |
| immunoglobulin octamer binding protein NF-A2; POU2F2 | |
| zinc finger protein 40 (ZNF40); human immunodeficiency virus type I | X51435 |
| enhancer-binding protein 1 (HIV-EP1); | |
| major histocompatibility complex binding protein 1 (MBP-1); | |
| positive regulatory domain II binding factor 1 (PRDII-BF1) | |
| nervous-system specific octamer-binding transcription factor N-oct3; | Z11933 |
| N-oct5A & N-oct5B; brain-specific homeobox/POU domain protein 2 (POU3F2); | |
| brn2; oct7 | |
| hypoxia-inducible factor 1 alpha (HIF1 alpha); ARNT-interacting protein; | U22431 |
| member of PAS protein 1 (MOP1) | |
| CCAAT/enhancer binding protein alpha (C/EBP alpha) | U34070 |
| HOMEOBOX PROTEIN MOX-2 (GROWTH ARREST-SPECIFIC HOMEOBOX) | X82629 |
| endothelial transcription factor GATA2 | M68891 |
| DNA-binding protein inhibitor Id-2 | M97796 |
| activating transcription factor 4 (ATF4); | D90209 |
| tax-responsive enhancer element B67 (TAXREB67); | |
| cAMP-response element-binding protein 2 (CREB2) | |
| heat shock factor protein 1 (HSF1); heat shock transcription factor 1 (HSTF1); TCF5 | M64673 |
| FK506-binding protein 13 precursor (FKBP13); | M65128 |
| FKBP2; peptidyl-prolyl cis-trans isomerase (PPIase) | |
| cAMP response element binding protein (CRE-BPI); transcription factor ATF2; HB16 | M31630 |
| cAMP-response element binding protein (CREB) | M34356 |
| early growth response protein 1 (EGR1); transcription factor ETR103; KROX24; | X52541; M62829 |
| zinc finger protein 225 (ZNF225); AT225 | |
| tristetraproline (TTP); TIS11; ZFP36; | M92843 |
| growth factor-inducible nuclear protein 475 (NUP475) | |
| purine-rich single-stranded DNA-binding protein alpha (PURA) | M96684 |
| transcription factor relB; I-rel | M83221 |
| CYCLIC-AMP-DEPENDENT TRANSCRIPTION FACTOR ATF-3 | L19871 |
| (ACTIVATING FACTOR 3) | |
| octamer-binding transcription factor 1 (oct-1; OTF1); | X13403 |
| octamer binding protein NF-A1; POU2F1 | |
| B-cell lymphoma 3-encoded protein (bcl-3) | M31732 |
| retinoic acid receptor gamma 1 (RAR-gamma 1; RARG) | M24857; M38258; M57707; |
| | M32074 |
| PRB-binding protein E2F1; retinoblastoma-binding protein 3 (RBBP3); | M96577 |
| retinoblastoma-associated protein 1 (RBAP1); PBR3 | |
| retinoic acid receptor alpha; retinoid X receptor alpha (RXRA) | X52773 |
| major histocompatibility complex enhancer-binding protein MAD3 | M69043 |
| fuse-binding protein 2 (FBP2) | U69126 |
| methyl CpG-binding protein 2 (MECP2) | L37298 |
| AP4 basic helix-loop-helix DNA-binding protein | S73885 |
| hepatocyte nuclear factor 4 (HNF4); transcription factor 14 | X76930 |
| metal-regulatory transcription factor | X78710 |
| cockayne syndrome group A; WD-repeat protein (CSA protein) | U28413 |
| RNase L inhibitor | X76388 |
| 40S ribosomal protein S5 | U14970 |
| glutamic-pyruvate transaminase 1 (GPT1); alanine aminotransferase 1 (AAT1) | D10355 |
| peptidylprolyl cis-trans isomerase A (PPIase; PPIA); rotamase; cyclophilin A (CYPA); | Y00052 |
| cyclosporin A-binding protein | |
| probable protein disulfide isomerase ER-60 precursor (ERP60); 58-kDa microsomal protein; | D16234; Z49835; D83485; |
| phospholipase C alpha | U42068 |
| HSC70-interacting protein; progesterone receptor-associated P48 protein | U28918 |
| chaperonin-containing T-complex polypeptide 1 beta subunit | AF026293 |
| (CCT-beta; CCTB; CCT2; TCP1-beta); 99D8.1 | |
| peroxisome assembly factor-2 (PAF-2); peroxisomal-type ATPase 1; peroxin-6; PEX6; | U56602 |
| PXAAA1 | |
| CELLULAR RETINOIC ACID BINDING PROTEIN | S74445 |
| endothelin-converting enzyme 1 | Z35307 |
| matrix metalloproteinase 14 precursor (MMP14); MMP-X1; membrane-type matrix metalloproteinase 1 (MT- | D26512; X83535 |
| MMP1) | |
| bleomycin hydrolase (BLM hydrolase) | X92106 |
| proteasome activator HPA28 subunit beta | D45248 |
| placental plasminogen activator inhibitor 2 (PAI-2; PLANH2); monocyte ARG-serpin; | M18082; J02685 |
| urokinase inhibitor | |
| alpha-2-macroglobulin precursor (alpha-2-M) | M11313 |
| tissue inhibitor of metalloproteinase 1 precursor (TIMP1); | X03124 |
| erythroid potentiating activity (EPA); fibroblast collagenase inhibitor | |
| alpha-1-antichymotrypsin precursor (ACT) | K01500 |
| alpha-1-antitrypsin precursor; alpha-1 protease inhibitor; alpha-1-antiproteinase | X02920 |
| DNA-binding protein A (DBPA); cold shock domain protein A (CSDA) | M24069 |
| decoy receptor 3 (DCR3) | AF104419 |
| T-complex protein 1 zeta-like subunit (CCT-zeta-like; TCP1-zeta-like); TSA303; | D78333 |
| testis-specific TCP20# | |
| chromatin assembly factor 1 p48 subunit (CAF1 p48 subunit); | X74262 |
| retinoblastoma-binding protein 4 (RBBP4); RBAP48; msil protein homolog | |
| high mobility group protein HMG2 | X62534 |
| DNA-binding protein UEV-1; UBE2V | U49278 |
| activator 1 140-kDa subunit (A1 140-kDa subunit); replication factor C large subunit; | L14922 |
| DNA-binding protein PO-GA | |
| replication factor C 36-kDa subunit (RFC36); activator 1 36-kDa subunit | L07540 |
| replication factor C 38-kDa subunit (RFC38); activator 1 38-kDa subunit | L07541 |
| replication protein A 70-kDa subunit (RPA70; REPA1; RF-A); | M63488 |
| single-stranded DNA-binding protein | |
| activator 1 40-kDa subunit (A1 40-kDa subunit); | M87338 |
| replication factor C 40-kDa subunit (RFC40); RFC2 | |
| activator 1 37-kDa subunit; replication factor C 37-kDa subunit (RFC37); RFC4 | M87339 |
| DNA topoisomerase I (TOP1) | J03250 |
| DNA topoisomerase II alpha (TOP2A) | J04088 |
| proliferating cyclic nuclear antigen (PCNA); cyclin | M15796; |
| | J04718 |
| DNA topoisomerase II beta (TOP2B) | X68060 |
| replication protein A 14-kDa subunit (RP-A) (RF-A); replication factor A protein 3 | L07493 |
| DNA nucleotidylexotransferase; terminal addition enzyme; | M11722; K01919 |
| terminal deoxynucleotidyltransferase (TDT); terminal transferase; DNTT | |
| DNA polymerase delta catalytic subunit | M80397 |
| DNA topoisomerase III (TOP3) | U43431 |
| excision repair cross-complementing rodent repair deficiency | L04791 |
| complementation group 6 (ERCC6); | |
| Cockayne syndrome protein 2 type B (CSB) | |
| xeroderma pigmentosum group G complementing protein (XPG); X-ray repair- | L20046; X69978 |
| complementing defective repair in Chinese hamster cells 5 (XRCC5) | |
| Ku (p70/p80) subunit; ATP-dependent DNA helicase II 86-kDa subunit; | M30938 |
| lupus ku autoantigen protein; thyroid-lupus autoantigen (TLAA); | |
| CTC box binding factor 85-kDa subunit (CTCBF; CTC85); nuclear factor IV | |
| xeroderma pigmentosum group B complementing protein (XPB); excision repair cross-complementing rodent | M31899 |
| repair deficiency complementation group 3 (ERCC3); | |
| basal transcription factor 2 89-kDa subunit (BTF2-p89; TFIIH 89-kDa subunit) | |
| Ku 70-kDa subunit; ATP-dependent DNA helicase II 70-kDa subunit; | M32865; S38729 |
| lupus ku autoantigen protein P70; thyroid-lupus auto-antigen (TLAA); | |
| CTC box binding factor 75-kDa subunit (CTC75) | |
| X-ray repair-complementing defective repair in Chinese hamster cells 1 (XRCC1) | M36089 |
| ubiquitin-conjugating enzyme E2 17-kDa (UBE2A); ubiquitin-protein ligase; | M74524 |
| ubiquitin carrier protein; HR6A | |
| DNA polymerase alpha catalytic subunit (POLA) | X06745 |
| 6-O-methylguanine-DNA methyltransferase (MGMT); methylated-DNA-protein- | M29971 |
| cysteine methyltransferase | |
| xeroderma pigmentosum group D complementing protein (XPD); X-ray repair- | X52221 |
| complementing defective repair in Chinese hamster cells 2 (XRCC2) | |
| excision repair cross-complementing rodent repair deficiency | M13194 |
| complementation group 1 (ERCC1) | |
| mutL protein homolog1 (MLH1); colon cancer nonpolyposis type 2 protein (COCA2) | U07418 |
| UV excision repair protein RAD23 homolog B (HHR23B); | D21090 |
| xeroderma pigmentosum group C repair complementing complex 58-kDa protein | |
| HHR23A; UV excision repair protein protein RAD23A | D21235 |
| DNA-dependent protein kinase (DNA-PK) + DNA-PK | U35835 + U47077 |
| catalytic subunit (DNA-PKCS) | |
| DNA damage repair & recombination protein 52 (RAD52) | U12134 |
| ataxia telangiectasia (ATM) | U33841 |
| RAD50 | U63139 |
| DNA ligase IV (LIG4); polydeoxyribonucleotide synthase | X83441 |
| DNA ligase III (LIG3); polydeoxyribonucleotide synthase | X84740 |
| DNA mismatch repair protein MSH2 | U04045; L47583 |
| DNA mismatch repair protein MSH6; mutS alpha 160-kDa subunit; | U54777 |
| G/T mismatch binding protein (GTMBP; GTBP) | |
| RecQ protein-like (DNA helicase Q1-like) | D37984 |
| DNA polymerase beta subunit (DPOB) | D29013 |
| DNA mismatch repair protein PMS1 (PMS1 protein homolog 1) | U13695 |
| DNA mismatch repair protein PMS2 (PMS1 protein homolog 2) | U13696 |
| ATP-dependent DNA ligase I (LIG1); polydeoxyribonucleotide synthase | M36067 |
| xeroderma pigmentosum group A complementing protein (XPA) | D14533 |
| damage-specific DNA binding protein p48 subunit (DDBB P48); implicated in xeroderma | U18300 |
| pigmentosum group E (DDB2) | |
| DNA repair protein XRCC4 | U40622 |
| G/T mismatch-specific thymine DNA glycosylase (TDG) | U51166 |
| DNA repair protein XRCC9 | U70310 |
| endonuclease III homolog 1; HNTH1; OCTS3 | U79718 |
| DNA-repair protein complementing XP-C cells; xeroderma pigmentosum group C | D21089 |
| complementing protein (p125) | |
| uracil-DNA glycosylase precursor (UNG1) | X15653 |
| DNA-(apurinic or apyrimidinic site) lyase; AP endonuclease 1 (APE1); | X59764; |
| apurinic/apyrimidinic endonuclease (APEX); APEX nuclease (APEN); | |
| REF1 | |
| X66133 | |
| DNA repair protein RAD54 homolog | X97795 |
| recA-like protein HsRad51; DNA repair protein RAD51 homolog | D13804 |
| V(D)J recombination activating protein 2 (RAG2) | M94633 |
| V(D)J recombination activating protein 1 (RAG1) | M29474 |
| muscle-specific DNase I-like precursor (DNase1L1; DNL1L); DNase X | X90392; L40817; U06846 |
| deoxyribonuclease I (DNase I) | M55983 |
| dual-specificity protein phosphatase 9; | Y08302 |
| mitogen-activated protein kinase phosphatase 4 | |
| (MAP kinase phosphatase 4 (MKP4) | |
| G1/S-specific cyclin D3 (CCND3) | M92287 |
| G1/S-specific cyclin D1 (CCND1); cyclin parathyroid adenomatosis 1 (PRAD1); | X59798 |
| bcl-1 oncogene | |
| G1/S-specific cyclin D2 (CCND2) + KIAK0002 | M90813 + D13639 |
| G2/mitotic-specific cyclin B1 (CCNB1) | M25753 |
| G1/S-specific cyclin E (CCNE) | M73812 |
| G2/mitotic-specific cyclin G1 (CCNG1; CYCG1) | U47413 |
| G1/S-specific cyclin C | M74091 |
| cyclin K | AF060515 |
| protein serine/threonine kinase STK1; cell division protein kinase 7 (CDK7); | L20320 |
| CDK-activating kinase (CAK); 39-kDa protein kinase | |
| cyclin-dependent protein kinase 2 (CDK2); p33 protein kinase | M68520 |
| extracellular signal-regulated kinase 2 (ERK2); | M84489 |
| mitogen-activated protein kinase 2 (MAP kinase 2; MAPK 2); p42-MAPK | |
| mitogen-activated protein kinase 3 (MAPK3; PRKM3); MAPK1; | X60188 |
| extracellular signal-regulated kinase 1 (ERK1); | |
| microtubule-associated protein 2 kinase; insulin-stimulated MAP2 kinase | |
| extracellular signal-regulated kinase 3 (ERK3); MAP kinase 3 (MAPK3; p97-MAPK); | X80692 |
| PRKM5 | |
| CDC-like kinase 3 (CLK3) | L29220 |
| cell division protein kinase 4; cyclin-dependent kinase 4 (CDK4); PSK-J3 | M14505 |
| extracellular signal-regulated kinase 5 (ERK5); BMK1 kinase | U25278 |
| cell division control protein 2 homolog (CDC2); p34 protein kinase; | X05360 |
| cyclin-dependent kinase 1 (CDK1) | |
| extracellular signal-regulated kinase 4 (ERK4); MAP kinase 4 (MAPK4; p63-MAPK); | X59727 |
| PRKM4 | |
| cell division protein kinase 5 (CDK5); tau protein kinase II catalytic subunit | X66364 |
| (TPKII catalytic subunit); serine/threonine protein kinase PSSALRE | |
| extracellular signal-regulated kinase 6 (ERK6); stress-activated protein kinase-3; | X79483 |
| mitogen-activated protein kinase p38 gamma; (MAP kinase p38 gamma) | |
| serine/threonine-protein kinase PLK1 (STPK13) | U01038 |
| checkpoint kinase 1 (CHK1) | AF016582 |
| aurora- & IPL1-like midbody-associated protein kinase 1 (AIM1); ARK2 | AF008552 |
| cyline G-associated kinase (GAK) | D88435 |
| special AT-rich sequence binding protein 1 (SATB1); MAR/SAR DNA-binding protein | M97287 |
| cyclin-dependent kinase inhibitor 1A (CDKN1A); | U09579; L25610 |
| melanoma differentiation-associated protein 6 (MDA6); | |
| CDK-interacting protein 1 (CIP1); WAF1; SDI1 | |
| wee1Hu CDK tyrosine 15-kinase; wee-1-like protein kinase | U10564 |
| cyclin-dependent kinase 4 inhibitor 2B (CDKN2B); p14-INK4B; | U17075; L36844 |
| multiple tumor suppressor 2 (MTS2) | |
| helix-loop-helix protein HLH 1R21; DNA-binding protein inhibitor Id-3; HEIR-1 | X69111 |
| DNA-binding protein inhibitor ID-1; Id-1H | D13889 |
| prothymosin alpha (PROT-alpha; PTMA) | M26708 |
| 40S ribosomal protein S19 (RPS19) | M81757 |
| p55CDC | U05340 |
| cell division cycle protein 25A (CDC25A); M-phase inducer phosphatase 1 | M81933 |
| CDC25B; CDC25HU2; M-phase inducer phosphatase 2 | M81934; S78187 |
| CDC25C; M-phase inducer phosphatase 3 | M34065 |
| growth inhibitory factor (GIF); metallothionein-III (MT-III; MT3) | D13365; |
| | M93311 |
| CDC37 homolog | U63131 |
| cell cycle protein P38-2G4 homolog; HG4-1 | U59435 |
| btg protein precursor; NGF-inducible anti-proliferative protein PC3 | U72649 |
| RCL growth-related c-myc-responsive gene | AF040105 |
| 40-kDa heat-shock protein 1 (HSP40); DNAJ protein homolog 1 (HDJ1; DNAJ1) | D49547 |
| 60-kDa heat shock protein (HSP60); HSPD1; 60-kDa chaperonin; | |
| mitochondrial matrix protein P1 precursor; p60 lymphocyte protein; | M34664 |
| HUCHA60; GROEL | |
| 90-kDa heat-shock protein A (HSP90A); HSP86; HSPCA | X07270 |
| 27-kDa heat-shock protein (HSP27); stress-responsive protein 27 (SRP27); | X54079 |
| estrogen-regulated 24-kDa protein; HSPB1 | |
| 70-kDa heat shock protein 1 (HSP70.1; HSPA1) | M11717 |
| heat shock 70-kDa protein 6 (heat shock 70-kDa protein B) | X51757; M11236 |
| heat shock cognate 71-kDa protein; heat shock 70-kDa protein 8 (HSPA8; HSC70); | Y00371 |
| HSP73 | |
| heat shock-related 70-kDa protein 2 | L26336 |
| major vault protein (MVP); lung resistance-related protein (LRP) | X79882 |
| thiosulfate sulfurtransferase; rhodanese | D87292 |
| soluble epoxide hydrolase (SEH); epoxide hydratase; cytosolic epoxide hydrolase (CEH); | L05779 |
| EPHX2 | |
| serum paraoxonase/arylesterase 1 (PON1); serum aryldiakylphosphatase 1; | |
| aromatic esterase 1 (A-esterase 1) | M63012 |
| polymorphic arylamine N-acetyltransferase (PNAT) + monomorphic (MNAT) | X14672; X17059 |
| quinone oxidoreductase; NADPH: quinone reductase; zeta-crystallin (CRYZ) | L13278; S58039 |
| cytosolic superoxide dismutase 1 (SOD1) | K00065; X02317 |
| cytochrome P450 IB1 (CYP1B1) | U03688 |
| cytochrome P450 IIA6 (CYP2A6) + CYP2A7 + CYP2A13 + CYP2A7PT + CYP2A7PC + U22029 + U22030 + U22044 | M33318; M33316 |
| cytochrome P450 IIB6 (CYP2B6) + CYP2B3 | M29874; J02864 |
| cytochrome P450 IIIA3 (CYP3A3) + CYP3A4 + CYP3A5 + CYP3A7 | L04751 |
| M13785 + M18907 + J04813 + D00408 | |
| cytochrome P450 IVA11 (CYP4A11) | |
| cytochrome P450 VIIA1 (CYP7A1) | X56088 |
| D-amino acid oxidase (DAMOX; DAO; DAAO) | X13227 |
| S-mephenytoin 4 hydroxylase; cytochrome P450 IIC9 (CYP2C9) + CYP2C10 + CYP2C17 + CYP2C18 + CYP2C19 | M21940 + M15331; |
| | M21939 + M61858 + |
| | M61854 |
| cytochrome P450 IIE1 (CYP2E1) | J02625 |
| cytochrome P450 IIF1 (CYP2F1) | J02906 |
| cytochrome P450 IVB1 (EC 1.14.14.1) (P450-HP) | J02871 |
| cytochrome P450 IA2 (P450-P3) (P450-4) | Z00036 |
| plasma glutathione peroxidase precursor (GPXP; GPX3) | D00632; X58295 |
| natural killer cell enhancing factor (NKEFB) + thiol-specific antioxidant protein | |
| (TSA); thioredoxin peroxidase 1 (TDPX1); | L19185 + Z22548; X82321 |
| thioredoxin-dependent peroxide reductase 1 | |
| thioredoxin peroxidase 2 (TDPX2); thioredoxin-dependent peroxide reductase 2; | X67951 |
| proliferation-associated gene (PAG); | |
| natural killer cell enhancing factor A (NKEFA) | |
| glutathione reductase (GRase; GSR; GR) | X15722 |
| microsomal glutathione S-transferase 12 (GST12; MGST1) | J03746; B28083 |
| glutathione S-transferase pi (GSTP1; GST3) | X08058; M24485 |
| glutathione peroxidase (GSHPX1; GPX1) | Y00483; M21304 |
| glutathione S-transferase theta 1 (GSTT1) | X79389 |
| methallothionein IH (MT1H); metallothinein0 | |
| (MT0) + MT1I; MT2 + MT1L + MT1R | X64177 + X97260 + |
| | X76717 + X97261 |
| glutathione peroxidase-gastrointestinal (GSHPX-GI); | X53463 |
| glutathione peroxidase-related protein 2 (GPRP) | |
| heme oxygenase 1 (HO1); HSOXYGR | X06985 |
| heme oxygenase 2 (HO2) | D21243; S34389 |
| dimethylaniline monooxygenase (N-oxide forming) 1 (EC 1.14.13.8); | M64082 |
| fetal hepatic flavin-containing monooxygenase 1 (FMO 1); | |
| dimethylaniline oxidase 1 | |
| glutathione S-transferase mu1 (GSTM1; GST1); HB subunit 4; GTH4 | X68676; S01719 |
| glutathione S-transferase A1 (GTH1; GSTA1); HA subunit 1; GST-epsilon | M25627 |
| glutathione-S-transferase (GST) homolog | U90313 |
| glutathione synthetase (GSH synthetase; GSH-S); glutathione synthase | U34683 |
| NAD(P)H dehydrogenase; quinone reductase; DT-diaphorase; | J03934 |
| azoreductase; phylloquinone reductase; menadione reductase | |
| growth arrest & DNA-damage-inducible protein (GADD45); | M60974 |
| DNA-damage-inducible transcript 1 (DDIT1) | |
| tumor necrosis factor alpha precursor (TNF-alpha; TNFA); cachectin | X01394 |
| lymphotoxin-alpha precursor (LT-alpha); tumor necrosis factor-beta (TNF-beta; TNFB) | D12614 |
| fas antigen ligand (FASL); apoptosis antigen ligand (APTL; APT1LG1); | D38122; |
| TNFSF6 | |
| U08137 | |
| tumor necrosis factor receptor (TNFR) + tumor necrosis factor receptor 2 (TNFR2); | M32315 + M55994 |
| tumor necrosis factor binding protein 2 (TBP2) | |
| tumor necrosis factor receptor 1 (TNFR1); tumor necrosis factor binding protein 1 | |
| (TBP1); CD120A antigen | M33294 |
| fasL receptor; apoptosis-mediating surface antigen fas; APO-1 antigen; CD95 antigen | M67454 |
| retinoic acid receptor beta (RXR-beta; RXRB) | M84820; X63522; S54072 |
| tumor necrosis factor receptor 1-associated death domain protein | |
| (TNFR1-associated death domain protein; TRADD) | L41690 |
| CD27BP (Siva) | U82938 |
| tumor necrosis factor type 1 receptor associated protein (TRAP1) | U12595 |
| caspase-2 precursor (CASP2); ICH-1L protease + ICH-1S protease | U13021 + U13022 |
| interleukin-1 beta convertase precursor (IL-1BC); IL-1 beta converting enzyme (ICE); | U13699; M87507; X65019 |
| p45; caspase-1 (CASP1) | |
| caspase-6 precursor (CASP6); cysteine protease MCH2 isoforms alpha + beta | U20536 + U20537 |
| caspase-4 precursor (CASP4); ICH-2 protease; TX protease; | U28014 + U28015 |
| ICE(REL)-II + caspase-5 precursor (CASP5); ICH-3 protease; TY protease; | |
| ICE(REL)-III | |
| caspase-7 precursor (CASP7); ICE-like apoptotic protease 3 (ICE-LAP3); | U37448 |
| apoptotic protease MCH-3; CMH-1 | |
| TNF-related apoptosis inducing ligand (TRAIL); APO-2 ligand (APO2L) | U57059 |
| caspase-8 precursor (CASP8); ICE-like apoptotic protease 5 (ICE-LAP5); | U60520; U58143; X98172; |
| | AF00962 |
| MORT1-associated CED-3 homolog (MACH); | |
| FADD-homologous ICE/CED-3-like protease (FADD-like ICE; FLICE); | |
| apoptotic cysteine protease MCH-5 | |
| apoptosis regulator bax | L22474 |
| apoptosis regulator bcl-x | Z23115; L20121; L20122 |
| apoptosis regulator bcl-2 | M14745 |
| NIP3 (NIP3) | U15174 |
| bcl2 homologous antagonist/killer (BAK) | U23765; U16811; X84213 |
| induced myeloid leukemia cell differentiation protein MCL-1 | L08246 |
| BAD protein; bcl-2 binding component 6 (BBC6); bcl-2L8 | U66879 |
| BCL-2 binding athanogene-1 (BAG-1); | S83171; Z35491 |
| glucocorticoid receptor-associated protein RAP46 | |
| interferon-inducible RNA-dependent protein kinase (P68 kinase) | M35663; U50648 |
| inducible nitric oxide synthase (INOS); type II NOS; hepatocyte NOS (HEP-NOS) | L09210 |
| defender against cell death 1 (DAD1) | D15057 |
| clusterin precursor (CLU); complement-associated protein SP-40; | M74816 |
| complement lysis inhibitor (CLI); apolipoprotein J (APOJ); | |
| testosterone-repressed prostate message 2 (TRPM2); | |
| sulfated glycoprotein 2 (SGP2) | |
| growth arrest & DNA-damage-inducible protein 153 (GADD153); | S40706; S62138 |
| DNA-damage-inducible transcript 3 (DDIT3); | |
| C/EBP homologous protein (CHOP) | |
| inhibitor of apoptosis protein1 (HIAP1; API1) + IAP homolog C; | U45878 + U37546 |
| TNFR2-TRAF signaling complex protein 1; MIHC | |
| cytoplasmic dynein light chain 1 (HDLC1); | U32944 |
| protein inihibitor of neuronal nitric oxide synthase (PIN) | |
| apoptosis inhibitor survivin | U75285 |
| sentrin; ubiquitin-like protein SMT3C; ubiquitin-homology domain protein PIC1; | U83117 |
| UBL1; SUMO-1; GAP modifying protein 1; GMP1 | |
| IEX-1L anti-death protein; PRG-1; DIF-2 | AF039067; AF071596 |
| poly(ADP-ribose) polymerase (PARP; PPOL ); ADPRT; | M18112; J03473 |
| NAD+ ADP-ribosyltransferase; poly(ADP-ribose) synthetase | |
| avian myelocytomatosis viral oncogene homolog (MYC) | V00568 |
| p53-associated mdm2 protein | Z12020; M92424 |
| platelet-derived growth factor B subunit precursor (PDGFB; PDGF2); | X02811; X02744; M12783; |
| | M16288 |
| bacaplermin; c-sis | |
| p53 cellular tumor antigen | M14694; |
| M14695 | |
| MYB-related protein B (B-MYB); avian myeloblastosis viral | |
| oncogene homolog-like 2 (MYBL2) | X13293 |
| triiodothyronine receptor; thyroid hormone receptor (THRA1); | M24898 |
| v-erbA-related protein ear-1 | |
| jun proto-oncogene; avian sarcoma virus 17 oncogene homolog; | J04111 |
| transcription factor AP-1 | |
| insulin-like growth factor binding protein 2 (IGFBP2) | M35410 |
| c-myc purine-binding transcription factor puf; nucleoside diphosphate kinase B | |
| (NDP kinase B; NDKB) + nm23-H2S | L16785 + M36981 |
| Abelson murine leukemia viral oncogene homolog 1 (ABL1) | M14752 |
| retinoblastoma-associated protein (RB1); PP110; P105-RB | M15400 |
| L-myc proto-oncogene (MYCL1) | M19720 |
| breast cancer type 2 susceptibility protein (BRCA2) | U43746 |
| fos-related antigen (FRA1); fosL1 | X16707 |
| nucleolar phosphoprotein B23; nucleophosmin (NPM); numatrin | M23613 |
| c-myc binding protein MM-1 | D89667 |
| c-fos proto-oncogene; G0S7 protein | K00650 |
| met proto-oncogene; hepatocyte growth factor receptor precursor (HGF-SF receptor) | J02958 |
| nucleoside diphosphate kinase A (NDKA); NDP kinase A; | X17620 |
| tumor metastatic process-associated protein; | |
| metastasis inhibition factor NM23 (NM23-H1) | |
| matrix metalloproteinase 11 (MMP11); stromelysin 3 | X57766 |
| box-dependent myc-interacting protein 1 | U68485 |
| H-ras proto-oncogene; transforming G protein | V00574 |
| protein-tyrosine phosphatase PTEN; mutated in multiple advanced cancers 1 (MMAC1); | U92436 |
| TEP1 | |
| prostaglandin G/H synthase 2 precursor (PGH synthase 2; PGHS2; PTGS2); | M90100 |
| cyclooxygenase 2 (COX2); prostaglandin-endoperoxide synthase 2 | |
| 78-kDa glucose regulated protein precursor (GRP 78); | M19645 |
| immunoglobulin heavy chain binding protein (BIP) | |
| complement 3 (C3) | K02765 |
| interleukin-10 precursor (IL-10); cytokine synthesis inhibitory factor (CSIF) | M57627 |
| thioredoxin (TRDX; TXN); ATL-derived factor (ADF); | J04026 |
| surface-associated sulphydrylprotein (SASP) | |
| enolase 1 alpha (ENO1); non-neural enolase (NNE); phosphopyruvate hydratase (PPH) | M14328 |
| biliverdin reductase A precursor (BLVRA; BVR) | U34877 |
| tyrosine aminotransferase (TAT); I-tyrosine: 2-oxoglutarateaminotransferase | X52520 |
| muscle-specific carbonic anhydrase III (CA3); carbonate dehydratase III | M29458 |
| spermidine/spermine N1-acetyltransferase (SSAT); | M55580 |
| diamine acetyltransferase; putrescine acetyltransferase | |
| L-lactate dehydrogenase H subunit (LDHB) | Y00711 |
| phosphoglyceride kinase 1 (PGK1; PGKA); primer recognition protein 2 (PRP2) | V00572 |
| glucose-6-phosphate dehydrogenase (G6PD) | X03674 |
| mitochondrial phosphoenolpyruvate carboxykinase 2 precursor (PEPCK-M; PCK2); | X92720 |
| phosphoenolpyruvate carboxylase | |
| galactoside 2-1-fucosyltransferase 2; | D87942 |
| GDP-1-fucose: beta-D-galactoside 2-alpha-1-fucosyltransferase 2; | |
| fucosyltransferase 2 (FUT2); secretor blood group alpha-2-fucosyltransferase; | |
| secretor factor 2 (SE2) | |
| galactosyltransferase-associated protein kinase p58 (GTA); | M37712 |
| cell division cycle 2-like 1 (CDC2L1; CLK1) | |
| adrenodoxin | M34788 |
| alcohol dehydrogenase alpha subunit + alcohol dehydrogenase 2 + alcohol | |
| dehydrogenase 3 | M12271 + D00137 + X04299 |
| alcohol dehydrogenase 5 chi polypeptide | M30471 |
| alcohol dehydrogenase class II pi subunit | M15943 |
| CREATINE KINASE B CHAIN | L47647 |
| fatty acid synthase | S80437 |
| hepatic triglyceride lipase (HTGL) | X07228 |
| bile-salt-activated lipase | M85201; M37044 |
| mitochondrial enoyl-CoA hydratase short subunit 1 | D13900 |
| peroxisomal bifunctonal enzyme | L07077 |
| peroxisomal acyl-CoA oxidase branched subunit (BRCOX) | X95190 |
| acyl-CoA dehydrogenase long chain-specific precursor (LCAD; ACADL) | M74096 |
| alcohol sulfotransferase | L20000 |
| estradiol 17 beta-dehydrogenase 1 | M36263 |
| cytochrome P450 XVIIA1 (CYP17A1) | M14564 |
| peroxisomal 3-ketoacyl-CoA thiolase precursor (PTHIO); | X14813 |
| peroxisomal 3-oxoacyl-CoA thiolase; beta-ketothiolase; | |
| acetyl-CoA acyltransferase (ACAA) | |
| 3-hydroxy-3-methylglutaryl-coenzyme A reductase (HMG-CoA reductase; HMGCR) | M11058 |
| lipoprotein lipase precursor (LPL) | M15856 |
| lung group IB phospholipase A2 precursor (PLA2); | M21054 |
| phosphatidylcholine 2-acylhydrolase | |
| mitochondrial cytochrome P450 XIA 1 precursor; P450(SCC); | M14565 |
| cholesterol side-chain cleavage enzyme; cholesterol desmolase CYP11A1 | |
| dihydrofolate reductase (DHFR) | V00507 |
| thymidylate synthase (TYMS; TS) | X02308 |
| cytosolic thymidine kinase (TK1) | K02581 |
| ribonucleoside-diphosphate reductase M1 subunit; ribonucleotide reductase | X59543 |
| microsomal UDP-glucuronosyltransferase 2B15 precursor (UDPGT); UDPGTH-3; | U08854; X63359; U06641; |
| UGT2B15 + microsomal 2B10 precursor (UDPGT); UGT2B10 + 2microsomal | J05428; Y00317 |
| B8 precursor | |
| GLCLC, GLCL (Glutamate-cysteine ligase catalytic subunit, | |
| gamma-glutamylcysteine synthetase) | M90656 |
| gamma-glutamyl hydrolase precursor (GGH; GH); folylpolygammaglutamyl hydrolase; | U55206 |
| gamma-glu-X carboxypeptidase; conjugase | |
| 3′-phosphoadenosine 5′-phosphosulfate synthase 1 (PAPS synthase 1; PAPSS1); | Y10387 |
| PAPS synthetase 1; sulfurylase kinase 1 (SK1) | |
| soluble glutamic oxaloacetic transaminase 1 (GOT1); | M37400 |
| cytoplasmic aspartate aminotransferase 1; transaminase A | |
| alcohol dehydrogenase 6 + aldehyde dehydrogenase 1 (ALDH1) | K03000 |
| peroxisomal acyl-coenzyme A oxidase | S69189 |
| very-long-chain-specific acyl-CoA dehydrogenase precursor (VLCAD) | D43682 |
| glutamate-cysteine ligase regulatory subunit (GLCLR); | P48507 |
| gamma-glutamylcysteine synthetase | |
| LOX (Protein-lysine 6-oxidase, Lysyl-Oxidase) | M94054 |
| ornithine decarboxylase | X16277 |
| corticosteroid 11-beta-dehydrogenase isozyme 2 | U14631 |
| cytochrome P450 VA1 (CYP5A1) | M80647 |
| mitochondrial aldehyde dehydrogenase precursor (class 2); ALDH1; ALDH2 | Y00109 |
| 5,6-dihydroxyindole-2-carboxylic acid oxidase precursor (DHICA oxidase); | X51420 |
| tyrosinase-related protein 1 (TRP-1); catalase B; glycoprotein-75 (GP75) | |
| tenascin precursor (TN); hexabrachion (HXB); cytotactin; neuronectin; GMEM; | X78565; M55618 |
| miotendinous antigen; glioma-associated extracellular matrix antigen | |
| OSTEOPONTIN PRECURSOR (BONE SIALOPROTEIN 1) | X13694 |
| ATP-binding cassette transporter (ABCR) | U88667 |
| tissue inhibitor of metalloproteinase 2 precursor (TIMP2) | J05593 |
| matrix metalloproteinase 15 (MMP15) | Z48482 |
| matrix metalloproteinase 14 (MMP14) | D26512 |
| matrix metalloproteinase 1 (MMP1) | X54925 |
| vinculin | M33308 |
| vimentin (VIM) | X56134; M14144 |
| serum amyloid A1 precursor (SAA1) | M23698 |
| senescence marker protein 30 (SMP30); regucalcin (RGN; RC) | D31815 |
| ubiquitin cross-reactive protein precursor (UCRP); | M13755 |
| alpha-inducible interferon; interferon-induced 17-kDa protein; G1P2; ISG15 | |
| laminin gamma 2 subunit precursor (LAMC2) | Z15009 |
| peroxisome assembly factor 1 (PAF1); | M86852 |
| peroxisomal membrane protein 3 (PXMP3; PMP3); | |
| 35-kDa peroxisomal membrane protein (PMP35); peroxin 2 (PEX2) | |
| peroxisomal membrane protein 69 (PMP69) | AF009746 |
| peroxisome biogenesis disorder protein 1 (PEX1) | AF026086 |
| mitochondrial glutamic oxaloacetic transaminase 2 (GOT2); | M22632 |
| aspartate aminotransferase 2; transaminase A | |
| nck, ash & phoshpholipase C gamma-binding protein (NAP4) | AB005216 |
| N-oxide-forming dimethylaniline monooxygenase 4; | Z11737 |
| hepatic flavin-containing monooxygenase 4 (FMO4) | |
| xeroderma pigmentosum group F complementing protein (XPF); | L77890 |
| DNA excision repair protein ERCC4; ERCC11 | |
| replication protein A 30-kDa subunit; replication factor A protein 4 (RPA4; RFA) | U24186 |
| mutY homolog (hMYH) | U63329 |
| beta crystallin A4 (CRYBA4) | U59057 |
| T-complex protein 1 epsilon subunit (TCP1-epsilon); CCT-epsilon (CCTE; CCT5) | D43950 |
| beta crystallin B1 (CRYBB1) | U35340 |
| beta crystallin B2 (CRYBB2); BP | L10035 |
| beta crystallin B3 (CRYBB3; CRYB3) | U71216 |
| mitochondrial 10-kDa heat shock protein (HSP10); | U07550 |
| 10-kDa chaperonin (CPN10); HSPE1 | |
| heat shock protein beta 3 (HSPB3); heat shock 17-kDa protein; HSPL27 | U15590 |
| probable protein disulfide isomerase P5 precursor | D49489 |
| 90-kDa heat-shock protein beta (HSP90); 84-kDa heat-shock protein beta (HSP84); | M16660 |
| HSPCB | |
| microsomal UDP-glucuronosyltransferase 1-6 precursor | |
| (UDPGT; UGT1.6; UGT1F; GNT1) | J04093 |
| glutathione S-transferase mu 3 (GSTM3); GST5 | J05459 |
| cytochrome P450 1A1 (CYP1A1); P450-P1; P450 form 6; P450-C | K03191 |
| peroxisome proliferator activated receptor alpha (PPAR-alpha; PPARA) | L02932 |
| protein disulfide isomerase-related protein precursor (PDIR) | D49490 |
| liver carboxylesterase precursor; acyl coenzyme A: cholesterol acyltransferase (ACAT); | L07765 |
| monocyte/macrophage serine esterase (hMSE); CES2 | |
| serum paraoxonase/arylesterase 3 (PON3); serum aryldiakylphosphatase 3; | L48516 |
| aromatic esterase 3 (A-esterase 3) | |
| cytochrome P450 XXIB (CYP21B); steroid 21-hydroxylase; CYP21A2 | M12792; M23280 |
| cytochrome P450 IID6 (CYP2D6); P450-DB1; debrisoquine 4-hydroxylase | M20403 |
| microsomal UDP-glucuronosyltransferase 1-1 precursor | |
| (UDPGT; UGT1.1; UGT1A; GNT1); bilirubin-specific isozyme 1 (hUG-BR1) | M57899 |
| microsomal UDP-glucuronosyltransferase 1-4 precursor | |
| (UDPGT; UGT1.4; UGT1D; GNT1); bilirubin-specific isozyme 2 (hUG-BR2) | M57951 |
| flavin-containing amine oxidase B (MAOB); monoamine oxidase | M69177 |
| eukaryotic peptide chain release factor subuni 1 (ERF1); TB3-1; C11 protein | M75715 |
| microsomal UDP-glucuronosyltransferase 1-3 precursor | |
| (UDPGT; UGT1.3; UGT1C; GNT1) | M84127 |
| structure-specific recognition protein 1 (SSRP1); | |
| recombination signal sequence recognition protein; T160 | M86737 |
| microsomal UDP-glucuronosyltransferase 1-2 precursor | |
| (UDPGT; UGT1.2; UGT1B; GNT1); HLUGP4 | S55985 |
| thiopurine S-methyltransferase (TPMT) | S62904 |
| meiotic recombination protein DMC1/LIM15 homolog | D63882 |
| short/branched chain-specific acyl-CoA dehydrogenase precursor (SBCAD; ACADSB); | U12778 |
| 2-methyl branched chain acyl-CoA dehydrogenase (2-MEBCAD) | |
| cytochrome P450 XIB1 precursor (CYP11B1); steroid 11-beta-hydroxylase (S11BH) | X55764 |
| cytochrome P450 IVA11 (CYP4A11) | X71480 |
| NADH-cytochrome B5 reductase (B5R); DIA1 | Y09501 |
| coproporphyrinogen III oxidase precursor (CPO); coproporphyrinogenase; | Z28409 |
| coprogen oxidase (COX) | |
| 110-kDa heat-shock protein (HSP110); 105-kDa heat-shock protein (HSP105); | D86956 |
| KIAA0201 | |
| gamma crystallin C (CRYGC; CRYG3; | U66582 + M11971; M11970 |
| gamma crystallin 2 + gamma crystallin B (CRYGB; CRYG2); | |
| gamma crystallin 1-2 | |
| heat shock transcription factor 4 (HHSF4) | D87673 |
| extracellular superoxide dismutase precursor (EC-SOD; SOD3) | J02947 |
| DNAJ protein homolog 2 (DNAJ2; hDJ2; HSJ2) | D13388 |
| DNA mismatch repair protein MSH3; divergent upstream protein (DUP); | J04810 |
| mismatch repair protein 1 (MRP1) | |
| protein disulfide isomerase-related protein ERP72 precursor | J05016 |
| replication protein A 32-kDa subunit (RPA32); | J05249 |
| replication factor A protein 2 (REPA2; RPA2; RFA) | |
| multidrug resistance-associated protein 1 (MRP1) | L05628 |
| calnexin precursor (CANX); | L10284; L18887; M94859; |
| | M98452 |
| major histocompatibility complex class I | |
| antigen-binding protein p88; IP90 | |
| cyclophilin-40 (CYP40; CYPD); 40-kDa peptidyl-prolyl cis-trans isomerase (PPIASE); | L11667 |
| rotamase; cyclophilin-related protein | |
| heat shock 70-kDa protein 4 (HSPA4); HSP70RY; | L12723 |
| heat shock 70-related protein APG-2 | |
| T-complex protein 1 theta subunit (TCP1-theta); | D13627 |
| CCT-theta (CCTQ; CCT8); KIAA0002 | |
| mitochondrial stress-70 protein precursor; 75-kDa glucose-regulated protein (GRP75); | L15189 |
| peptide-binding protein 74 (PBP74); mortalin (MOT); HSPA9B | |
| p23; 23-kDa progesterone receptor-associated protein | L24804; L24805 |
| FLAP endonuclease 1 (FEN1); maturation factor 1 (MF1) | L37374 |
| FK506-binding protein 12 (FKBP12); peptidyl-prolyl cis-trans isomerase (PPIase); | M34539; M80199; M92423; |
| rotamase | X55741; X52220 |
| heat shock factor protein 2 (HSF2); heat shock transcription factor 2 (HSTF2) | M65217 |
| 3-methyladenine DNA glycosylase (ADPG); 3-alkyladenine DNA glycosylase; | M74905 |
| N-methylpurine-DNA glycosirase (MPG) | |
| calreticulin precursor (CRP55); calregulin; HACBP; ERP60; | M84739 |
| 52-kDa ribonucleoprotein autoantigen RO/SS-A | |
| transformation-sensitive protein IEF SSP 3521 | M86752 |
| alpha crystallin B subunit (alpha(B)-crystallin; CRYAB; CRYA2); | S45630 |
| Rosenthal fiber component | |
| heat shock protein beta 2 (HSPB2); DMPK-binding protein; MKBP | S67070 |
| alpha crystallin A chain (CRYAA; CRYA1) | U05569 |
| nicotinamide N-methyltransferase (NNMT) | U08021 |
| phenol-sulfating phenol sulfotransferase 1 (PPST1); | U09031 + U28170 + L19956 |
| thermostable phenol sulfotransferase (TS-PST); | |
| HAST1/HAST2; ST1A3; STP1 + PPST2; ST1A2; | |
| STP2 + monoamine-sulfating phenol sulfotransferase | |
| NADP + dihydropyrimidine dehydrogenase precursor (DPD); | U09178 |
| dihydrouracil dehydrogenase; dihydrothymine dehydrogenase (DPYD) | |
| transcriptional regulator atrX; X-linked helicase II (XH2); | U09820 |
| X-linked nuclear protein (XNP); RAD54L | |
| 26S proteasome regulatory subunit S2 (PSMD2); | U12596 |
| tumor necrosis factor type 1 receptor-associated protein (TRAP2); 55.11 protein | |
| damage-specific DNA-binding protein p127 subunit (DDBA p127); DDB1 | U18299 |
| T-complex protein 1 delta subunit (TCP1-delta); CCT-delta (CCTD; CCT4); | U38846 |
| stimulator of tar RNA binding (SRB) | |
| 7,8-dihydro-8-oxoguanine triphosphatase (8-oxo-dGTPase); mutT homolog 1 (MTH1) | D16581 |
| 150-kDa oxygen-regulated protein ORP150 | U65785 |
| 48-kDa FKBP-associated protein (FAP48) | U73704 |
| T-complex protein 1 eta subunit (TCP1-eta); CCT-eta (CCTH; CCT7); | U83843 |
| HIV-1 NEF interacting protein | |
| catalase (CAT) | X04076 |
| porphobilinogen deaminase (PBGD); hydroxymethylbilane synthase (HMBS); | X04808 |
| pre-uroporphyrinogen synthase | |
| Mn+ superoxide dismutase 2 precursor (SOD2) | X07834; X59445 |
| 94-kDa glucose-regulated protein (GRP94); endoplasmin precursor; GP96 homolog; | X15187; M33716 |
| tumor rejection antigen 1 (TRA1) | |
| uracil-DNA glycosylase 2 (UNG2) | X52486 |
| T-complex protein 1 alpha subunit (TCP1-alpha); CCT-alpha (CCTA; CCT1) | X52882 |
| 40S ribosomal protein S3 (RPS3) | X55715 |
| 47-kDa heat shock protein precursor; collagen-binding protein 1 (CBP1); | X61598 + D83174 |
| colligin 1 + collagen binding protein 2 (CBP2) | |
| T-complex protein 1 gamma subunit (TCP1-gamma); | X74801; U17104 |
| CCT-gamma (CCTG; CCT3); TRIC5 | |
| transcription factor IIH (TFIIH); 52-kDa basic transcription factor 2 subunit (BTF2p52) | Y07595 |
| x-ray repair cross-complementing protein 2 (XRCC2) | Y08837 |
| 8-oxyguanine DNA glycosylase 1 (OGG1); mutM homolog (MMH) | Y11838 |
| 34-kDa basic transcription factor 2 subunit (BTF2p34) | Z30093 |
| N-oxide forming dimethylaniline monooxygenase 5; | L37080 |
| hepatic flavin-containing monooxygenase 5 (FMO5); dimethylaniline oxidase 5 | |
| ubiquitin-like protein NEDD8 | D23662 |
| multidrug resistance protein 3 (MDR3); P-glycoprotein 3 (PGY3) | M23234 |
| ubiqitin-conjugating enzyme E2-17-kDa (UBE2B); ubiquitin-protein ligase; | M74525 |
| ubiquitin carrier protein; HR6B | |
| p59 protein; HSP-binding immunophilin (HBI); possible peptidyl-prolyl cis-trans isomerase (PPIase); | M88279 |
| rotamase; 52-kDa FK506-binding protein (FKBP52); | |
| FKBP59; HSP56; FKBP4 | |
| heat shock protein 40 homolog (HSP40 homolog); DNAJW | U40992 |
| 51-kDa FK506-binding protein (FKBP51); peptidyl-prolyl cis-trans isomerase (PPIase); | U42031 |
| rotamase; 54-kDa progesterone receptor-associated immunophilin; | |
| FKBP54; FF1 antigen; HSP90-binding immunophilin | |
| hematopoietic progenitor kinase (HPK1) | U66464 |
| SPS1/Ste20 homolog KHS1 U77129 | |
| liver glyceraldehyde 3-phosphate dehydrogenase (GAPDH; G3PDH) | X01677 |
| brain-specific tubulin alpha 1 subunit (TUBA1) | K00558 |
| HLA class I histocompatibility antigen C-4 alpha subunit (HLAC) | M11886 |
| cytoplasmic beta-actin (ACTB) | X00351 |
| 23-kDa highly basic protein; 60S ribosomal protein L13A (RPL13A) | X56932 |
| 40S ribosomal protein S9 | U14971 |
| ubiquitin | M26880 |
| phospholipase A2 | M86400 |
| hypoxanthine-guanine phosphoribosyltransferase (HPRT) | V00530 |

3. A method for the toxicological diagnosis according to claim 1, further characterized in that the methylation state or the methylation pattern of essentially all of the genes listed in claim 2 is investigated.

4. The method according to claim 1, further characterized in that a set of genes is preferably investigated for methylation in which up to 25% of the genes listed in claim 2 are not contained.

5. The method according to claim 1, further characterized in that at least 95% of the genes listed in claim 2 are investigated for their methylation state or their methylation pattern, together with a limited number of additional unlisted genes.

6. The method according to claim 1, further characterized in that up to 25% of the listed genes are replaced by a complete set of other unlisted genes.

7. The method according to claim 1 for a set of genes according to one or more of claims 1-6, in which the chemically pretreated DNA sequence of the genes to be detected coincides to at least 95% with the correspondingly pretreated DNA sequence of the genes from the above list.

8. The method for toxicological diagnosis according to one of claims 1-7, further characterized in that the following steps are conducted:
a) a sample that contains the DNA of an organism or a cell culture is taken from this organism or cell culture;
b) in a sample which contains the genomic DNA, cytosine bases that are unmethylated at the 5-position are converted by chemical treatment to uracil or thymidine, while cytosine bases that are methylated at the 5-position remain unchanged;
c) fragments from this chemically pretreated genomic DNA are amplified;
d) the amplified fragments are hybridized to (probe) oligonucleotides or PNA oligomers;
e) the methylation state or the methylation pattern is derived from the hybridization behavior of the amplified fragments;
f) the effect of a substance on the organism or the cell culture is concluded by comparison with data of the methylation states of other samples and/or the effect of this substance on the organism or the cell culture is compared with other substances from a toxicological point of view.

9. The method according to claim 8, further characterized in that the chemical treatment is conducted with the solution of a bisulfite (=disulfite, hydrogen sulfite).

10. The method according to claim 8, further characterized in that the amplification is conducted by means of the polymerase chain reaction (PCR).

11. The method according to claim 10, further characterized in that sets of primer oligonucleotides comprising at least two oligonucleotides are used for the amplification, each of whose sequences corresponds to a segment that is at least 18 base pairs long of nucleic acids obtained after chemical modification of the genes listed in claim 2 or is complementary to these sequences.

12. The method according to claim 10 or 11, further characterized in that sets of primer oligonucleotides are used, which contain identifiable labels.

13. The method according to claim 12, further characterized in that the labels of the primer oligonucleotides are fluorescent labels.

14. The method according to claim 12, further characterized in that the labels of the primer oligonucleotides are radionuclides.

15. The method according to claim 12, further characterized in that the labels of the primer oligonucleotides are removable molecular fragments with typical masses, which can be detected in a mass spectrometer.

16. The method according to claim 8, further characterized in that the hybridized amplificates, fragments of the amplificates or probes that are complementary to the amplificates, are detected in the mass spectrometer.

17. The method according to claim 15 or 16, further characterized in that the produced fragments have a single positive or single negative net charge for better detectability in the mass spectrometer.

18. The method according to one of claims 8 to 17, further characterized in that the (probe) oligonucleotides or PNA oligomers according to claim 8d) are for the most part-identical or complementary to a sequence segment that is at least 9 nulceotides long of at least one of the genes according to claim 2, as it is present after the chemical modification according to claim 8 and—contain at least one CG or TG dinucleotide.

19. The method according to claim 18, further characterized in that oligonucleotides are used, in which the cytosine of the CpG dinucleotide or the thymine of the TpG dinucleotide is the 5th to 9th nucleotide from the 5-end of the 13-mer.

20. The method according to claim 18, further characterized in that PNA oligomers are used, in which the cytosine of the CpG dinucleotide or the thymine of the TpG dinucleotide is the 4th to 6th nucleotide from the 5-end of the 9-mer.

21. The method according to claim 8, further characterized in that more than ten different fragments are amplified and are investigated with the same set of probe oligonucleotides according to one of claims 18 to 20.

22. The method according to claim 8, further characterized in that the probe oligonucleotides or PNA oligomers are bound to defined sites of a solid phase.

23. The method according to claim 22, further characterized in that different detection oligonucleotides and/or PNA oligomer sequences are arranged on a planar solid phase in the form of a rectangular or hexagonal grid.

24. The method according to claim 22 or 23, further characterized in that the labels that are introduced on the amplificates at each position of the solid phase at which an oligonucleotide is found can be identified.

25. The method according to one of claims 22 to 24, further characterized in that the solid-phase surface is preferably comprised of silicon, glass, polystyrene, aluminum, steel, iron, copper, nickel, silver, or gold.

26. The method according to one of the preceding claims, wherein the genomic DNA has been obtained from a sample containing DNA, whereby sources for DNA include, e.g., cell lines, biopsies, blood, sputum, stool, urine, cerebrospinal fluid, tissue embedded in paraffin, for example, tissue from eyes, intestine, kidney, brain, heart, prostate, lungs, breast or liver, histological slides and all possible combinations thereof.

27. The method according to one of the preceding claims for the diagnosis and/or prognosis of adverse events for patients or individuals, whereby these adverse events are related to the diagnosis of toxicologically important parameters.

28. Use of a method according to one of the preceding claims for the diagnosis and/or prognosis of adverse events for patients or individuals, whereby these adverse events are related to the diagnosis of toxicologically important parameters.

29. A kit containing
a) a chemical substance for modification of cytosine bases;
b) primer oligonucleotides for the amplification of modified nucleic acids;
c) probe oligonucleotides or PNA oligomers;
and optionally
d) instructions for conducting a method according to one of the preceding claims;
e) control nucleic acid with known methylation state or methylation pattern.
